(19) United States
(12) Patent Application Publication
(10) Pub. No.: US 2024/0262930 A1
(43) Pub. Date: Aug. 8, 2024

(54) MULTI-SPECIFIC ANTIBODY CONSTRUCTS AGAINST THE MUC1-C/EXTRACELLULAR DOMAIN (MUC1-C/ED)

(71) Applicants: XYONE THERAPEUTICS INC., Canton, MA (US); DANA-FARBER CANCER INSTITUTE, INC., Boston, MA (US)

(72) Inventors: Surender KHARBANDA, Natick, MA (US); Govindaswamy PANCHAMOORTHY, Marlborough, MA (US); Donald W. KUFE, Wellesley, MA (US)

(73) Assignees: XYONE THERAPEUTICS INC., Canton, MA (US); DANA-FARBER CANCER INSTITUTE, INC., Boston, MA (US)

(21) Appl. No.: 18/561,413

(22) PCT Filed: May 27, 2022
(86) PCT No.: PCT/US2022/031431

§ 371 Date: Nov. 16, 2023

Related U.S. Application Data
(60) Provisional application No. 63/194,597, filed on May 28, 2021.

(51) Int. Cl. C07K 16/30; A61K 45/06; A61K 47/68; A61K 47/69; A61P 35/02; C07K 16/28; C07K 16/32

(52) U.S. Cl. CPC C07K 16/3092; A61K 45/06; A61K 47/6849; A61K 47/6889; A61K 47/6913; A61P 35/02; C07K 16/2809; C07K 16/2818; C07K 16/283; C07K 16/2851; C07K 16/2863; C07K 16/2896; C07K 16/32; C07K 2317/24; C07K 2317/31; C07K 2317/35; C07K 2317/565; C07K 2317/732; C07K 2317/734

(57) ABSTRACT

The present disclosure is directed to multispecific antibody constructs binding to MUC1-C/extracellular domain (MUC1-C/ECD) and to at least’ one other binding target, wherein the binding target comprising CD3 (cluster of differentiation 3). Also provided are methods of using such constructs to treat cancers that express the MUC1 antigen. Further disclosed are sequences of recombinant multispecific antibodies.

### PRIORITY CLAIM

This application claims benefit of priority to U.S. Provisional Application Ser. No. 63/194,597, filed May 28, 2021, the entire contents of which are hereby incorporated by reference.

### REFERENCE TO SEQUENCE LISTING

The instant application contains a Sequence Listing, which has been submitted in ASCII format via EFS-Web and is hereby incorporated by reference in its entirety. Said ASCII copy, created on May 26, 2022, is named GENU0048WO_ST25.txt and is 112 KB in size.

### BACKGROUND

### 1. Field

The present disclosure relates generally to the fields of medicine, oncology and immunotherapeutics. More particularly, it concerns the development of multi-specific immunoreagents for use in treating MUC1-positive cancers.

### 2. Related Art

Mucins are extensively O-glycosylated proteins that are predominantly expressed by epithelial cells. The secreted and membrane-bound mucins form a physical barrier that protects the apical borders of epithelial cells from damage induced by toxins, microorganisms and other forms of stress that occur at the interface with the external environment. The transmembrane mucin 1 (MUC1) can also signal to the interior of the cell. MUC1 has no sequence similarity with other membrane-bound mucins, except for the presence of a sea urchin sperm protein-enterokinase-agrin (SEA) domain (Duraisamy et al., 2006). In that regard, MUC1 is translated as a single polypeptide and then undergoes autocleavage at the SEA domain Macao, 2006).

MUC1 has been studied extensively by the inventors and others for its role in cancer. As discussed above, human MUC1 is a heterodimeric glycoprotein, translated as a single polypeptide and cleaved into N- and C-terminal subunits (MUC1-N and MUC1-C) in the endoplasmic reticulum (Ligtenberg et al., 1992; Macao et al., 2006; Levitin et al., 2005). Aberrant overexpression of MUC1, as found in most human carcinomas (Kufe et al., 1984), confers anchorage-independent growth and tumorigenicity (Li et al., 2003a; Huang et al., 2003; Schroeder et al., 2004; Huang et al., 2005). Other studies have demonstrated that overexpression of MUC1 confers resistance to apoptosis induced by oxidative stress and genotoxic anti-cancer agents (Yin and Kufe, 2003; Ren et al., 2004; Raina et al., 2004; Yin et al., 2004; Raina et al., 2006; Yin et al., 2007).

The family of tethered and secreted mucins functions in providing a protective barrier of the epithelial cell surface. With damage to the epithelial layer, the tight junctions between neighboring cells are disrupted, and polarity is lost as the cells initiate a heregulin-induced repair program (Vermeer et al., 2003). MUC1-N is shed from the cell surface (Abe and Kufe, 1989), leaving MUC1-C to function as a transducer of environmental stress signals to the interior of the cell. In this regard, MUC1-C forms cell surface complexes with members of the ErbB receptor family, and MUC1-C is targeted to the nucleus in the response to heregulin stimulation (Li et al., 2001; Li et al., 2003c). MUC1-C also functions in integrating the ErbB receptor and Wnt signaling pathways through direct interactions between the MUC1 cytoplasmic domain (CD) and members of the catenin family (Huang et al., 2005; Li et al., 2003c; Yamamoto et al., 1997; Li et al., 1998; Li et al., 2001; Li and Kufe, 2001). Other studies have demonstrated that MUC1-CD is phosphorylated by glycogen synthase kinase 3β, c-Src, protein kinase C6, and c-Abl (Raina et al., 2006; Li et al., 1998; Li et al., 2001; Ren et al., 2002). Inhibiting any of the foregoing interactions represents a potential point of therapeutic intervention for MUC1-related cancers.

### SUMMARY

Thus, in accordance with the present disclosure, there is provided a recombinant antibody construct that binds selectively to MUC1-C extracellular domain (MUC1-C/ECD) defined by SEQ ID NO: 2, wherein said antibody construct also binds to:
(a) CD3;
(b) CD16;
(c) CD28;
(d) myeloid specific antigen;
(e) ErbB2;
(f) EGFR;
(g) CD3 and PD1;
(h) CD16 and PD1;
(i) CD47;
(j) SIRPα;
(k) NKG2D,
(1) Siglec 9.
   The antibody construct may be divalent, trivalent or tetravalent. The antibody construct may have two distinct binding specificities for MUC1-C-/ECD. The antibody construct may have MUC1 binding specificity arising from heavy CDR1, CDR2 and CDR3 sequences of SEQ ID NOs: 3, 5, and 7, respectively, and light chain CDR1, CDR2 and CDR3 sequences of SEQ ID NOS; 4, 6, and 8, respectively, and/or MUC1 binding specificity arising from heavy CDR1, CDR2 and CDR3 sequences of SEQ ID NOs: 9, 11, and 13, respectively, and light chain CDR1, CDR2 and CDR3 sequences of SEQ ID NOS; 10, 12, and 14, respectively.

The antibody construct may contain one or more mutations permitting two distinct antibody chains to lock. The antibody construct may contain IgG sequences and/or may be a humanized version of a murine antibody, such as a humanized antibody construct containing IgG sequences. The antibody construct may further comprise a label, such as a peptide tag, an enzyme, a magnetic particle, a chromophore, a fluorescent molecule, a chemilluminescent molecule, or a dye. The antibody construct may further comprise an antitumor drug linked thereto, such as where the antitumor drug is linked to said antibody construct through a photolabile linker or an enzymatically-cleaved linker. The antitumor drug may be a toxin, a radioisotope, a cytokine or an enzyme.

The antibody construct may comprise a sequence of SEQ ID NOS: 22-42. The antibody construct may comprise a sequence having 80%, 85%, 90%, 95% or 99% homology to SEQ ID NOS: 22-42. The antibody construct may be conjugated to a nanoparticle or a liposome. Induction of cell death may comprise antibody-dependent cell cytotoxicity or complement-mediated cytoxocity.

Also provided is a method of treating cancer comprising contacting a MUC1-positive cancer cell in a subject with the antibody construct as defined herein. The MUC1-positive cancer cell may be a solid tumor cell, such as a lung cancer cell, brain cancer cell, head & neck cancer cell, breast cancer cell, skin cancer cell, liver cancer cell, pancreatic cancer cell, stomach cancer cell, colon cancer cell, rectal cancer cell, uterine cancer cell, cervical cancer cell, ovarian cancer cell, testicular cancer cell, skin cancer cell, or esophageal cancer cell. The MUC1-positive cancer cell may be a leukemia or myeloma, such as acute myeloid leukemia, chronic myelogenous leukemia or multiple myeloma.

The method may further comprise contacting said MUC1-positive cancer cell with a second anti-cancer agent or treatment, such as where said second anti-cancer agent or treatment is selected from chemotherapy, radiotherapy, immunotherapy, hormonal therapy, or toxin therapy. The second anti-cancer agent or treatment may inhibit an intracellular MUC1 function. The second anti-cancer agent or treatment may be given at the same time as said antibody construct or may be given before and/or after said antibody construct. The MUC1-positive cancer cell may be a metastatic cancer cell, a multiply drug resistant cancer cell or a recurrent cancer cell. The antibody construct may result in the induction of cell death, such as by antibody-dependent cell cytotoxicity or complement-mediated cytoxocity.

Also provided cell expressing an antibody construct as described herein.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-N: Schematic of various forms of bi-specific antibodies. (FIG. 1A) h3D1-hCD3 bi-specific antibody (construct pair ‘A’). Bi-specific DNA constructs were generated (Construct A) to make a homodimer of bi-valent hMUC1-C(h3D1 clone) and bi-valent human CD3 (hCD3) binding paratopes. h3D1 (VH-CH1)-hFc-hCD3 (VL-VH)+h3D1 (VL-CL). The inventors have also generated LALA-PG mutations to abolish any Fc receptor mediated effector mechanism (SEQ ID NOS: 30+31). (FIG. 1B) h7B8-1-hCD3 bi-specific antibody (construct pair ‘B’). The inventors have generated a monomer containing a separate light chain of h7B8-1 antibody. A h7B8-1-hCD3 bi-specific constructs were generated to have a single MUC1-C binding site by incorporating a monomeric Fc that has better stability and does not dimerize (SEQ ID NOS; 32+33). (FIG. 1C) h3D1-hCD3 bi-specific antibody (construct pair ‘C’). The inventors have generated a heterodimer with scFvs brought together via knob-into-hole binding. This construct has bivalent binding site for MUC1-C and monovalent binding site for CD3 due to heterodimerization by using knobs-into-hole technology with the indicated mutations (T366S, T368A, Y407V against T366W) in the Fc region. The knobs-into-hole technology applies large amino acids in one chain to create a “knob” and employs smaller amino acids for a corresponding “hole” in the other chain. In addition, electrostatic steering of two oppositely charged heavy chains in combination with the single chain variable fragment (scFv) technology ensures correct chain assembly (SEQ ID NOS: 22+23). (FIG. 1D) h3D1-hCD3 bi-specific antibody (scFv) (construct ‘D’). This format of bi-specific antibody has a single chain variable fragment (scFv) that has one binding site each for MUC1-C and CD3 and remains as a monomer due to the indicated mutations (SEQ ID NO: 22). (FIG. 1E) h3D1-hCD3-hPD-1 tri-specific antibody (construct pair ‘E’). This format employs the same strategy of heterodimerization as in FIG. 1C, but it includes a binding site for PD-1 (SEQ ID NOS: 22+34). (FIG. 1F) h3D1-hCD3-hPD-1 tri-specific antibody (construct pair ‘F’). This format employs the strategy of heterodimerization as in FIG. 1C, but it includes a binding site for PD-1 as well as with different orientations of heavy and light chains for h3D1 and hPD-1 (SEQ ID NOS: 24+35). (FIG. 1G) h7B8-1-hCD3-hPD-1 tri-specific antibody (construct pair ‘G’). This format employs the same strategy of heterodimerization as in FIG. 1C, but it includes a binding site for PD-1 (SEQ ID NOS: 26+36). (FIG. 1H) h7B8-1-hCD3-hPD-1 tri-specific antibody (construct pair ‘H’). This format employs the strategy of heterodimerization as in FIG. 1C, but it includes a binding site for PD-1 as well as with different orientations of heavy and light chains for h7B8-1 and hPD-1 (SEQ ID NOS: 28+37). (FIG. 1I) h7B8-1-hCD3 bi-specific antibody (construct pair ‘I’). The inventors have generated a heterodimer with scFvs brought together via knob-into-hole binding. This construct has bivalent binding site for MUC1-C and monovalent binding site for CD3 due to heterodimerization by using knobs-into-hole technology with the indicated mutations (T366S, T368A, Y407V against T366W) in the Fc region. The knobs-into-hole technology applies large amino acids in one chain to create a “knob” and employs smaller amino acids for a corresponding “hole” in the other chain. In addition, electrostatic steering of two oppositely charged heavy chains in combination with the single chain variable fragment (scFv) technology ensures correct chain assembly (SEQ ID NOS: 26+27). (FIG. 1J) h7B8-1-hCD3 bi-specific antibody (scFv) (construct ‘J’). This format of bi-specific antibody has a single chain variable fragment (scFv) that has one binding site each for MUC1-C and CD3 and remains as a monomer due to the indicated mutations (SEQ ID NO: 26). (FIGS. 1K-N) Bi-paratopic bi-specific-MUC1-C/CD3 constructs in four different designs.

FIG. 2: Purification of bi-specific antibodies. All the indicated constructs were expressed in CHO-K1 cells and single cell clones of each bispecific format were generated. Cells from the clones were expanded, suspension cultures were maintained, and the bispecific antibodies purified using protein A columns. Purified proteins were checked by SDS-PAGE. Lanes 1-3 contain the indicated bispecific proteins in reducing conditions. Lanes 4-6 contain the same proteins in non-reducing conditions. A=h3D1(VH-CH1)-hFc-hCD3(VL-VH)+h3D1(VL-CL); B=h7B8-1(VH-CH1)-mhFc-hCD3(VL-VH)+h7B8-1(VL-CL); D=h3D1(VH-VL)-hFc-hCD3(VL-VH)-scFv.

FIG. 3: Assessment of bispecific antibody binding to the MUC1-C antigen on ZR-75-1 hormone dependent breast cancer cells by flow cytometry. Cells were incubated with 4 ug/ml of test antibody or an IgG1 isotype control antibody for 60 minutes followed by appropriate secondary antibody. Antibody binding to the cell surface was analyzed using flow cytometry. Binding of h3D1-hCD3 bispecific antibody to cell surface MUC1-C on breast adenocarcinoma cell line ZR75-1. Isotype matched human IgG1 and h3D1 were used as negative and positive control respectively for the binding.

FIG. 4: Assessment of bispecific antibody construct binding to CD3 on Jurkat T cell line by flow cytometry. Binding of h3D1-hCD3 bispecific antibody construct to CD3 on a T cell line, Jurkat. Isotype matched human IgG1 and anti-hCD3 were used as negative and positive control respectively for the binding.

FIGS. 5A-C: T cell activation by bispecific antibodies. Target cells well were plated in growth medium in a 96 well plate and incubated overnight. Varying concentrations of bispecific antibodies (indicated) were added to cells followed by TCR/CD3 effector cells (NFAT-Jurkat) and incubated for 6 hrs. Bio-Glo™ reagent was added and luminescence was quantified using Molecular Devices FilterMax F5 reader. Data were fitted to a 4PL curve using GraphPad Prism software. (FIG. 5A) ZR-75-1, breast adenocarcinoma cells (10,000 cells/well) treated with indicated bispecific antibodies starting from 20 μg/ml with 2-fold serial dilutions and NFAT-Jurkat, 100,000 cells/well. (FIG. 5B) ZR-75-1, breast adenocarcinoma cells (40,000 cells/well) treated with indicated bispecific antibodies starting from 30 μg/ml with 3-fold serial dilutions and NFAT-Jurkat, 100,000 cells/well. (FIG. 5C) HCT116 expressing MUC1 (HCT/MUC1) or the vector (HCT116/Vector) cells (10,000 cells/well) treated with indicated bispecific antibodies starting from 10 μg/ml with 3-fold serial dilutions and NFAT-Jurkat, 100,000 cells/well.

### DESCRIPTION OF ILLUSTRATIVE EMBODIMENTS

The inventors have generated multi-specific antibody constructs with binding specificity to a 58 amino acid non-shed portion of the external domain of the MUC1-C protein as well as to at least one and optionally two other binding targets. Such constructs can also be engineered to have binding specificity to multiple MUC1-C epitopes. These antibodies have ben demonstrated the ability to stimulate T-cells and therefore are useful in treatment of MUC1 related cancers. These and other aspects of the disclosure are described in greater detail below.

### I. MUC1

### A. Structure

MUC1 is a mucin-type glycoprotein that is expressed on the apical borders of normal secretory epithelial cells (Kufe et al., 1984). MUC1 forms a heterodimer following synthesis as a single polypeptide and cleavage of the precursor into two subunits in the endoplasmic reticulum (Ligtenberg et al., 1992). The cleavage may be mediated by an autocatalytic process (Levitan et al., 2005). The >250 kDa MUC1 N-terminal (MUC1-N) subunit contains variable numbers of 20 amino acid tandem repeats that are imperfect with highly conserved variations and are modified by O-linked glycans (Gendler et al., 1988; Siddiqui et al., 1988). MUC1-N is tethered to the cell surface by dimerization with the ˜23 kDa C-terminal subunit (MUC1-C), which includes a 58 amino acid extracellular region, a 28 amino acid transmembrane domain (underline) and a 72-amino acid cytoplasmic domain (CD; bold) (Merlo et al., 1989). It is the 58 amino acid portion of the MUC1-C/ECD (italics) to which antibodies of the present disclosure bind. The human MUC1-C sequence is shown below:

(SEQ ID NO: 1)
SVVVQLTLAFREGTINVHDVETQFNQYKTEAASRYNLTISDVSVS
DVPFPFSAQSGAGVPGWGIALLVLVCVLVALAIVYLIALAVCQCR
RKNYGQLDIFPARDTYHPMSEYPTYHTHGRYVPPSSTDRSPYEKV
SAGNGGSSLSYTNPAVAATSANL

The bold sequence indicates the CD, and the underlined portion is an oligomer-inhibiting peptide. With transformation of normal epithelia to carcinomas, MUC1 is aberrantly overexpressed in the cytosol and over the entire cell membrane (Kufe et al., 1984; Perey et al., 1992). Cell membrane-associated MUC1 is targeted to endosomes by clathrin-mediated endocytosis (Kinlough et al., 2004). In addition, MUC1-C, but not MUC1-N, is targeted to the nucleus (Baldus et al., 2004; Huang et al., 2003; Li et al., 2003a; Li et al., 2003b; Li et al., 2003c; Wei et al., 2005; Wen et al., 2003) and mitochondria (Ren et al., 2004).

### B. Function

MUC1-C interacts with members of the ErbB receptor family (Li et al., 2001b; Li et al., 2003c; Schroeder et al., 2001) and with the Wnt effector, β-catenin (Yamamoto et al., 1997). The epidermal growth factor receptor and c-Src phosphorylate the MUC1 cytoplasmic domain (MUC1-CD) on Y-46 and thereby increase binding of MUC1 and β-catenin (Li et al., 2001a; Li et al., 2001b). Binding of MUC1 and β-catenin is also regulated by glycogen synthase kinase 3β and protein kinase C6 (Li et al., 1998; Ren et al., 2002). MUC1 colocalizes with β-catenin in the nucleus (Baldus et al., 2004; Li et al., 2003a; Li et al., 2003c; Wen et al., 2003) and coactivates transcription of Wnt target genes (Huang et al., 2003). Other studies have shown that MUC1 also binds directly to p53 and regulates transcription of p53 target genes (Wei et al., 2005). Notably, overexpression of MUC1-C is sufficient to induce anchorage-independent growth and tumorigenicity (Huang et al., 2003; Li et al., 2003b; Ren et al., 2002; Schroeder et al., 2004).

### II. PRODUCING MONOCLONAL ANTIBODIES

### A. General Methods

Antibodies to the MUC1-C/ECD may be produced by standard methods as are well known in the art (see, e.g., Antibodies: A Laboratory Manual, Cold Spring Harbor Laboratory, 1988; U.S. Pat. No. 4,196,265). The methods for generating monoclonal antibodies (MAbs) generally begin along the same lines as those for preparing polyclonal antibodies. The first step for both these methods is immunization of an appropriate host or identification of subjects who are immune due to prior natural infection. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. One particular murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line. More recently, additional fusion partner lines for use with human B cells have been described, including KR12 (ATCC CRL-8658; K6H6/B5 (ATCC CRL-1823 SHM-D33 (ATCC CRL-1668) and HMMA2.5 (Posner et al., 1987). The antibodies in this disclosure were generated using the SP2/0/mIL-6 cell line, an IL-6 secreting derivative of the SP2/0 line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like.

The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

Antibodies according to the present disclosure may be defined, in the first instance, by their binding specificity, which in this case is for MUC1-C/ECD, and in particular: SVVVQLTLAFREGTINVHDVETQFNQYKTEAASRYNLTISDVSVSDVPFPFSAQSGAG (SEQ ID NO: 2). Those of skill in the art, by assessing the binding specificity/affinity of a given antibody using techniques well known to those of skill in the art, can determine whether such antibodies fall within the scope of the instant claims.

In on embodiment, the antibody construct retains Immunoglobulin G (IgG) antibody isotype sequences. Representing approximately 75% of serum immunoglobulins in humans, IgG is the most abundant antibody isotype found in the circulation. IgG molecules are synthesized and secreted by plasma B cells. There are four IgG subclasses (IgG1, 2, 3, and 4) in humans, named in order of their abundance in serum (IgG1 being the most abundant). The range from having high to no affinity for the Fc receptor.

IgG is the main antibody isotype found in blood and extracellular fluid allowing it to control infection of body tissues. By binding many kinds of pathogens-representing viruses, bacteria, and fungi-IgG protects the body from infection. It does this via several immune mechanisms: IgG-mediated binding of pathogens causes their immobilization and binding together via agglutination; IgG coating of pathogen surfaces (known as opsonization) allows their recognition and ingestion by phagocytic immune cells; IgG activates the classical pathway of the complement system, a cascade of immune protein production that results in pathogen elimination; IgG also binds and neutralizes toxins. IgG also plays an important role in antibody-dependent cell-mediated cytotoxicity (ADCC) and intracellular antibody-mediated proteolysis, in which it binds to TRIM21 (the receptor with greatest affinity to IgG in humans) in order to direct marked virions to the proteasome in the cytosol. IgG is also associated with Type II and Type III Hypersensitivity. IgG antibodies are generated following class switching and maturation of the antibody response and thus participate predominantly in the secondary immune response. IgG is secreted as a monomer that is small in size allowing it to easily perfuse tissues. It is the only isotype that has receptors to facilitate passage through the human placenta. Along with IgA secreted in the breast milk, residual IgG absorbed through the placenta provides the neonate with humoral immunity before its own immune system develops. Colostrum contains a high percentage of IgG, especially bovine colostrum. In individuals with prior immunity to a pathogen, IgG appears about 24-48 hours after antigenic stimulation.

In addition, the presently claimed antibodies will have at least a secondary binding specificity, namely, binding to CD3, CD16, myeloid specific antigen, EGFR, ErbB2, TILs, CD3/PD1 or CD16/PD1. In another aspect, the antibodies may be defined by the sequences that determine their binding specificity. Sequences are provided in the Examples that follow blow.

Particular examples of antibodies that are employed with the present disclosure are those designated as 7B8-1 and 3D1, the CDRs for which are set out in Table 1.

TABLE 1
Antibody Construct CDR Sequences

| Original | | |
|---|---|---|
| Antibody | Heavy Chain | Light Chain |
| GO-702 | GFTF | CRASESVQ |
| (7B8-1) CDR1 | NYFW | YSGTSLMH |
| | SEQ ID NO: 3 | SEQ ID NO: 4 |
| GO-702 | ILPGTGST | GASNVET |
| (7B8-1) CDR2 | SEQ ID NO: 5 | SEQ ID NO: 6 |
| GO-702 | RYDYTSSMDY | QQNWKVPWT |
| (7B8-1) CDR3 | SEQ ID NO: 7 | SEQ ID NO: 8 |
| 3D1 CDR1 | NFWMN | RASQSIGTSIH |
| | SEQ ID NO: 9 | SEQ ID NO: 10 |
| 3D1 CDR2 | QIYPGDGD | YASE |
| | TNYNGKFKG | SIS |
| | SEQ ID NO: 11 | SEQ ID NO: 12 |
| 3D1 CDR3 | SYYRSAWFAY | QQSNNWPLT |
| | SEQ ID NO: 13 | SEQ ID NO: 14 |

Furthermore, the antibodies sequences may vary from the sequences provided above, optionally using methods discussed in greater detail below. For example, amino sequences may vary from those set out above in that (a) the variable regions may be segregated away from the constant domains of the light chains, (b) the amino acids may vary from those set out while not drastically affecting the chemical properties of the residues thereby (so-called conservative substitutions), (c) the amino acids may vary from those set out above by a given percentage, e.g., 80%, 85%, 90%, 91%, 92%, 93%, 94%, 95%, 96%, 97%, 98% or 99% homology. Alternatively, the nucleic acids encoding the antibodies may (a) be segregated away from the constant domains of the light chains, (b) vary from those set out above while not changing the residues coded thereby, (c) may vary from those set out above by a given percentage, e.g., 70%, 75%, 80%, 85%, 90%, 91%, 92%, 93%, 94%, 95%, 96%, 97%, 98% or 99% homology, or (d) vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C.

In making conservative changes in amino acid sequence, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include arginine and lysine; glutamate and aspartate; serine and threonine; glutamine and asparagine; and valine, leucine and isoleucine.

### C. Engineering of Antibody Constructs

In various embodiments, one may choose to engineer sequences of the identified antibodies for a variety of reasons, such as improved expression, improved cross-reactivity, diminished off-target binding or abrogation of one or more natural effector functions, such as activation of complement or recruitment of immune cells (e.g., T cells). In particular, IgM antibodies may be converted to IgG antibodies. The following is a general discussion of relevant techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns. Recombinant full-length IgG antibodies can be generated by subcloning heavy and light chain Fv DNAs from the cloning vector into a Lonza pConIgG1 or pConK2 plasmid vector, transfected into 293 Freestyle cells or Lonza CHO cells, and collected and purified from the CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

pCon Vectors™ are an easy way to re-express whole antibodies. The constant region vectors are a set of vectors offering a range of immunoglobulin constant region vectors cloned into the pEE vectors. These vectors offer easy construction of full length antibodies with human constant regions and the convenience of the GS System™.

It may be desirable to “humanize” antibodies produced in non-human hosts in order to attenuate any immune reaction when used in human therapy. Such humanized antibodies may be studied in an in vitro or an in vivo context. Humanized antibodies may be produced, for example by replacing an immunogenic portion of an antibody with a corresponding, but non-immunogenic portion (i.e., chimeric antibodies). PCT Application PCT/US86/02269; EP Application 184,187; EP Application 171,496; EP Application 173,494; PCT Application WO 86/01533; EP Application 125,023; Sun et al. (1987); Wood et al. (1985); and Shaw et al. (1988); all of which references are incorporated herein by reference. General reviews of “humanized” chimeric antibodies are provided by Morrison (1985); also incorporated herein by reference. “Humanized” antibodies can alternatively be produced by CDR or CEA substitution. Jones et al. (1986); Verhoeyen et al. (1988); Beidler et al. (1988); all of which are incorporated herein by reference.

The present disclosure also contemplates isotype modification. By modifying the Fc region to have a different isotype, different functionalities can be achieved. For example, changing to IgG₄ can reduce immune effector functions associated with other isotypes.

Modified antibodies may be made by any technique known to those of skill in the art, including expression through standard molecular biological techniques, or the chemical synthesis of polypeptides. Methods for recombinant expression are addressed elsewhere in this document.

### D. Expression

Nucleic acids according to the present disclosure will encode antibodies, optionally linked to other protein sequences. As used in this application, the term “a nucleic acid encoding a MUC1-C antibody construct” refers to a nucleic acid molecule that has been isolated free of total cellular nucleic acid. In certain embodiments, the disclosure concerns antibodies that are encoded by any of the sequences set forth herein.

TABLE 2
CODONS

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The DNA segments of the present disclosure include those encoding biologically functional equivalent proteins and peptides of the sequences described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

Within certain embodiments, expression vectors are employed to express a MUC1-C ligand trap in order to produce and isolate the polypeptide expressed therefrom. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

### 1. Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment.

A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Table 3 lists several elements/promoters that may be employed, in the context of the present disclosure, to regulate the expression of a gene. This list is not intended to be exhaustive of all the possible elements involved in the promotion of expression but, merely, to be exemplary thereof. Table 4 provides examples of inducible elements, which are regions of a nucleic acid sequence that can be activated in response to a specific stimulus.

TABLE 3
Promoter and/or Enhancer

| Promoter/Enhancer | References |
|---|---|
| Immunoglobulin Heavy Chain | Banerji et al., 1983; Gilles et al., 1983; Grosschedl et |
| | al., 1985; Atchinson et al., 1986, 1987; Imler et al., |
| | 1987; Weinberger et al., 1984; Kiledjian et al., 1988; |
| | Porton et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et |
| | al., 1986; Kadesch et al., 1986; Wang et al., 1986; |
| | Ondek et al., 1987; Kuhl et al., 1987; Schaffner et al., |
| | 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et al., |
| | 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987; Glue et |
| | al., 1988 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; Jakobovits |
| | et al., 1988; Feng et al., 1988; Takebe et al., 1988; |
| | Rosen et al., 1988; Berkhout et al., 1989; Laspia et |
| | al., 1989; Sharp et al., 1989; Braddock et al., 1989 |
| Cytomegalovirus (CMV) | Weber et al., 1984; Boshart et al., 1985; Foecking et |
| | al., 1986 |
| Gibbon Ape Leukemia Virus | Holbrook et al., 1987; Quinn et al., 1989 |

TABLE 4
Inducible Elements

| Element | Inducer | References |
|---|---|---|
| MT II | Phorbol Ester (TFA) | Palmiter et al., 1982; |
| | Heavy metals | Haslinger et al., 1985; Searle |
| | | et al., 1985; Stuart et al., |
| | | 1985; Imagawa et al., 1987, |
| | | Karin et al., 1987; Angel et |
| | | al., 1987b; McNeall et al., |
| | | 1989 |
| MMTV (mouse mammary | Glucocorticoids | Huang et al., 1981; Lee et |
| tumor virus) | | al., 1981; Majors et al., |
| | | 1983; Chandler et al., 1983; |
| | | Lee et al., 1984; Ponta et al., |
| | | 1985; Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T Antigen | Taylor et al., 1989, 1990a, |
| | | 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et aa. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996). Tumor specific promoters also will find use in the present disclosure. Some such promoters are set forth in Table 5.

TABLE 5
CANDIDATE TISSUE-SPECIFIC PROMOTERS
FOR CANCER GENE THERAPY

| | Cancers in which promoter is | Normal cells in which |
|---|---|---|
| Tissue-specific promoter | active | promoter is active |
| Carcinoembryonic antigen | Most colorectal carcinomas; | Colonic mucosa; |
| (CEA)* | 50% of lung carcinomas; 40- | gastric mucosa; lung |
| | 50% of gastric carcinomas; | epithelia; eccrine |
| | most pancreatic carcinomas; | sweat glands; cells in |
| | many breast carcinomas | testes |
| Prostate-specific antigen | Most prostate carcinomas | Prostate epithelium |
| (PSA) | | |
| Vasoactive intestinal peptide | Majority of non-small cell | Neurons; lymphocytes; |
| (VIP) | lung cancers | mast cells; eosinophils |
| Surfactant protein A (SP-A) | Many lung adenocarcinomas | Type II pneumocytes; |
| | cells | Clara |
| Human achaete-scute | Most small cell lung cancers | Neuroendocrine cells |
| homolog (hASH) | | in lung |
| Mucin-1 (MUC1)** | Most adenocarcinomas | Glandular epithelial |
| | (originating from any tissue) | cells in breast and in |
| | | respiratory, |
| | | gastrointestinal, and |
| | | genitourinary tracts |
| Alpha-fetoprotein | Most hepatocellular | Hepatocytes (under |
| | carcinomas; possibly many | certain conditions); |
| | testicular cancers | testis |
| Albumin | Most hepatocellular | Hepatocytes |
| | carcinomas | |
| Tyrosinase | Most melanomas | Melanocytes; |
| | | astrocytes; Schwann |
| | | cells; some neurons |
| Tyrosine-binding protein | Most melanomas | Melanocytes; |
| (TRP) | | astrocytes, Schwann |
| | | cells; some neurons |
| Keratin 14 | Presumably many squamous | Keratinocytes |
| | cell carcinomas (e.g., Head | |
| | and neck cancers) | |
| EBV LD-2 | Many squamous cell | Keratinocytes of upper |
| | carcinomas of head and neck | digestive |
| | | Keratinocytes of upper |
| | | digestive tract |
| Glial fibrillary acidic protein | Many astrocytomas | Astrocytes |
| (GFAP) | | |
| Myelin basic protein (MBP) | Many gliomas | Oligodendrocytes |
| Testis-specific angiotensin- | Possibly many testicular | Spermatazoa |
| converting enzyme (Testis- | cancers | |
| specific ACE) | | |
| Osteocalcin | Possibly many osteosarcomas | Osteoblasts |
| E2F-regulated promoter | Almost all cancers | Proliferating cells |
| HLA-G | Many colorectal carcinomas; | Lymphocytes; |
| | many melanomas; possibly | monocytes; |
| | many other cancers | spermatocytes; |
| | | trophoblast |
| FasL | Most melanomas; many | Activated leukocytes: |
| | pancreatic carcinomas; most | neurons; endothelial |
| | astrocytomas possibly many | cells; keratinocytes; |
| | other cancers | cells in |
| | | immunoprivileged |
| | | tissues; some cells in |
| | | lungs, ovaries, liver, |
| | | and prostate |
| Myc-regulated promoter | Most lung carcinomas (both | Proliferating cells |
| | small cell and non-small cell); | (only some cell-types): |
| | most colorectal carcinomas | mammary epithelial |
| | | cells (including non- |
| | | proliferating) |
| MAGE-1 | Many melanomas; some non- | Testis |
| | small cell lung carcinomas; | |
| | some breast carcinomas | |
| VEGF | 70% of all cancers | Cells at sites of |
| | (constitutive overexpression in | neovascularization |
| | many cancers) | (but unlike in tumors, |
| | | expression is transient, |
| | | less strong, and never |
| | | constitutive) |
| bFGF | Presumably many different | Cells at sites of |
| | cancers, since bFGF | ischemia (but unlike |
| | expression is induced by | tumors, expression is |
| | ischemic conditions | transient, less strong, |
| | | and never constitutive) |
| COX-2 | Most colorectal carcinomas; | Cells at sites of |
| | many lung carcinomas; | inflammation |
| | possibly many other cancers | |
| IL-10 | Most colorectal carcinomas; | Leukocytes |
| | many lung carcinomas; many | |
| | squamous cell carcinomas of | |
| | head and neck; possibly many | |
| | other cancers | |
| GRP78/BiP | Presumably many different | Cells at sites of |
| | cancers, since GRP7S | ishemia |
| | expression is induced by | |
| | tumor-specific conditions | |
| CarG elements from Egr-1 | Induced by ionization | Cells exposed to |
| | radiation, so conceivably most | ionizing radiation; |
| | tumors upon irradiation | leukocytes |

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

### 2. IRES

In certain embodiments of the disclosure, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

### 3. Multi-Purpose Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

### 4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, herein incorporated by reference).

### 5. Termination Signals

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

### 6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

### 7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

### 8. Selectable and Screenable Markers

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

### 9. Viral Vectors

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

Adenoviral Vectors. In particular embodiments, an adenoviral expression vector is contemplated for the delivery of expression constructs. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear, double-stranded DNA, with a genome ranging from 30 to 35 kb in size (Reddy et al., 1998; Morrison et al., 1997; Chillon et al., 1999). An adenovirus expression vector according to the present disclosure comprises a genetically engineered form of the adenovirus. Advantages of adenoviral gene transfer include the ability to infect a wide variety of cell types, including non-dividing cells, a mid-sized genome, ease of manipulation, high infectivity and the ability to be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a V sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral infection, the MLP is activated, resulting in the expression of the late (L) genes, encoding polypeptides required for adenovirus encapsidation. The intermediate region encodes components of the adenoviral capsid. Adenoviral inverted terminal repeats (ITRs; 100-200 bp in length), are cis elements, and function as origins of replication and are necessary for viral DNA replication. The V sequence is required for the packaging of the adenoviral genome.

A common approach for generating adenoviruses for use as a gene transfer vectors is the deletion of the E1 gene (E1⁻), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the E1 promoter or a heterologous promoter. The E1⁻, replication-deficient virus is then proliferated in a “helper” cell line that provides the E1 polypeptides in trans (e.g., the human embryonic kidney cell line 293). Thus, in the present disclosure it may be convenient to introduce the transforming construct at the position from which the E1-coding sequences have been removed. However, the position of insertion of the construct within the adenovirus sequences is not critical to the disclosure. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. Nos. 5,670,488; 5,932,210, each specifically incorporated herein by reference).

Although adenovirus based vectors offer several unique advantages over other vector systems, they often are limited by vector immunogenicity, size constraints for insertion of recombinant genes and low levels of replication. The preparation of a recombinant adenovirus vector deleted of all open reading frames, comprising a full length dystrophin gene and the terminal repeats required for replication (Haecker et al., 1996) offers some potentially promising advantages to the above mentioned adenoviral shortcomings. The vector was grown to high titer with a helper virus in 293 cells and was capable of efficiently transducing dystrophin in mdx mice, in myotubes in vitro and muscle fibers in vivo. Helper-dependent viral vectors are discussed below.

A major concern in using adenoviral vectors is the generation of a replication-competent virus during vector production in a packaging cell line or during gene therapy treatment of an individual. The generation of a replication-competent virus could pose serious threat of an unintended viral infection and pathological consequences for the patient. Armentano et al. (1990), describe the preparation of a replication-defective adenovirus vector, claimed to eliminate the potential for the inadvertent generation of a replication-competent adenovirus (U.S. Pat. No. 5,824,544, specifically incorporated herein by reference). The replication-defective adenovirus method comprises a deleted E1 region and a relocated protein IX gene, wherein the vector expresses a heterologous, mammalian gene.

Other than the requirement that the adenovirus vector be replication defective, or at least conditionally defective, the nature of the adenovirus vector is not believed to be crucial to the successful practice of the disclosure. The adenovirus may be of any of the 42 different known serotypes and/or subgroups A-F. Adenovirus type 5 of subgroup C is the preferred starting material in order to obtain the conditional replication-defective adenovirus vector for use in the present disclosure. This is because adenovirus type 5 is a human adenovirus about which a great deal of biochemical and genetic information is known, and it has historically been used for most constructions employing adenovirus as a vector.

As stated above, the typical vector according to the present disclosure is replication defective and will not have an adenovirus E1 region. Adenovirus growth and manipulation is known to those of skill in the art, and exhibits broad host range in vitro and in vivo (U.S. Pat. Nos. 5,670,488; 5,932,210; 5,824,544). This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. Many experiments, innovations, preclinical studies and clinical trials are currently under investigation for the use of adenoviruses as gene delivery vectors. For example, adenoviral gene delivery-based gene therapies are being developed for liver diseases (Han et al., 1999), psychiatric diseases (Lesch, 1999), neurological diseases (Smith, 1998; Hermens and Verhaagen, 1998), coronary diseases (Feldman et al., 1996), muscular diseases (Petrof, 1998), gastrointestinal diseases (Wu, 1998) and various cancers such as colorectal (Fujiwara and Tanaka, 1998; Dorai et al., 1999), pancreatic, bladder (Irie et al., 1999), head and neck (Blackwell et al., 1999), breast (Stewart et al., 1999), lung (Batra et al., 1999) and ovarian (Vanderkwaak et al., 1999).

Retroviral Vectors. In certain embodiments of the disclosure, the uses of retroviruses for gene delivery are contemplated. Retroviruses are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

The retroviral genome and the proviral DNA have three genes: gag, pol, and env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication.

A recombinant retrovirus of the present disclosure may be genetically modified in such a way that some of the structural, infectious genes of the native virus have been removed and replaced instead with a nucleic acid sequence to be delivered to a target cell (U.S. Pat. Nos. 5,858,744; 5,739,018, each incorporated herein by reference). After infection of a cell by the virus, the virus injects its nucleic acid into the cell and the retrovirus genetic material can integrate into the host cell genome. The transferred retrovirus genetic material is then transcribed and translated into proteins within the host cell. As with other viral vector systems, the generation of a replication-competent retrovirus during vector production or during therapy is a major concern. Retroviral vectors suitable for use in the present disclosure are generally defective retroviral vectors that are capable of infecting the target cell, reverse transcribing their RNA genomes, and integrating the reverse transcribed DNA into the target cell genome, but are incapable of replicating within the target cell to produce infectious retroviral particles (e.g., the retroviral genome transferred into the target cell is defective in gag, the gene encoding virion structural proteins, and/or in pol, the gene encoding reverse transcriptase). Thus, transcription of the provirus and assembly into infectious virus occurs in the presence of an appropriate helper virus or in a cell line containing appropriate sequences enabling encapsidation without coincident production of a contaminating helper virus.

The growth and maintenance of retroviruses is known in the art (U.S. Pat. Nos. 5,955,331; 5,888,502, each specifically incorporated herein by reference). Nolan et al. describe the production of stable high titre, helper-free retrovirus comprising a heterologous gene (U.S. Pat. No. 5,830,725, specifically incorporated herein by reference). Methods for constructing packaging cell lines useful for the generation of helper-free recombinant retroviruses with amphoteric or ecotrophic host ranges, as well as methods of using the recombinant retroviruses to introduce a gene of interest into eukaryotic cells in vivo and in vitro are contemplated in the present disclosure (U.S. Pat. No. 5,955,331).

Currently, the majority of all clinical trials for vector-mediated gene delivery use murine leukemia virus (MLV)-based retroviral vector gene delivery (Robbins et al., 1998; Miller et al., 1993). Disadvantages of retroviral gene delivery include a requirement for ongoing cell division for stable infection and a coding capacity that prevents the delivery of large genes. However, recent development of vectors such as lentivirus (e.g., HIV), simian immunodeficiency virus (SIV) and equine infectious-anemia virus (EIAV), which can infect certain non-dividing cells, potentially allow the in vivo use of retroviral vectors for gene therapy applications (Amado and Chen, 1999; Klimatcheva et al., 1999; White et al., 1999; Case et al., 1999). For example, HIV-based vectors have been used to infect non-dividing cells such as neurons (Miyatake et al., 1999), islets (Leibowitz et al., 1999) and muscle cells (Johnston et al., 1999). The therapeutic delivery of genes via retroviruses are currently being assessed for the treatment of various disorders such as inflammatory disease (Moldawer et al., 1999), AIDS (Amado and Chen, 1999; Engel and Kohn, 1999), cancer (Clay et al., 1999), cerebrovascular disease (Weihl et al., 1999) and hemophilia (Kay, 1998).

Herpesviral Vectors. Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons). Similar to adenovirus, HSV also can infect a variety of cell types including muscle (Yeung et al., 1999), ear (Derby et al., 1999), eye (Kaufman et al., 1999), tumors (Yoon et al., 1999; Howard et al., 1999), lung (Kohut et al., 1998), neuronal (Garrido et al., 1999; Lachmann and Efstathiou, 1999), liver (Miytake et al., 1999; Kooby et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discernable phases or kinetic classes. These phases of genes are referred to as the Immediate Early (IE) or a genes, Early (E) or β genes and Late (L) or γ genes. Immediately following the arrival of the genome of a virus in the nucleus of a newly infected cell, the IE genes are transcribed. The efficient expression of these genes does not require prior viral protein synthesis. The products of IE genes are required to activate transcription and regulate the remainder of the viral genome.

For use in therapeutic gene delivery, HSV must be rendered replication-defective. Protocols for generating replication-defective HSV helper virus-free cell lines have been described (U.S. Pat. Nos. 5,879,934; 5,851,826, each specifically incorporated herein by reference in its entirety). One IE protein, ICP4, also known as α4 or Vmw175, is absolutely required for both virus infectivity and the transition from IE to later transcription. Thus, due to its complex, multifunctional nature and central role in the regulation of HSV gene expression, ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma (Hayashi et al., 1999), intracerebral and intradermal tumors (Moriuchi et al., 1998), B-cell malignancies (Suzuki et al., 1998), ovarian cancer (Wang et al., 1998) and Duchenne muscular dystrophy (Huard et al., 1997).

Adeno-Associated Viral Vectors. Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

The use of helper virus, however, presents a number of problems. First, the use of adenovirus in a rAAV production system causes the host cells to produce both rAAV and infectious adenovirus. The contaminating infectious adenovirus can be inactivated by heat treatment (56° C. for 1 hour). Heat treatment, however, results in approximately a 50% drop in the titer of functional rAAV virions. Second, varying amounts of adenovirus proteins are present in these preparations. For example, approximately 50% or greater of the total protein obtained in such rAAV virion preparations is free adenovirus fiber protein. If not completely removed, these adenovirus proteins have the potential of eliciting an immune response from the patient. Third, AAV vector production methods which employ a helper virus require the use and manipulation of large amounts of high titer infectious helper virus, which presents a number of health and safety concerns, particularly in regard to the use of a herpesvirus. Fourth, concomitant production of helper virus particles in rAAV virion producing cells diverts large amounts of host cellular resources away from rAAV virion production, potentially resulting in lower rAAV virion yields.

Lentiviral Vectors. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al., (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene, such as the STAT-1α gene in this disclosure, into that packaging cell yields a producer cell which releases infectious viral particles carrying the foreign gene of interest. The env preferably is an amphotropic envelope protein which allows transduction of cells of human and other species.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence, such as the STAT-1α encoding polynucleotide sequence herein, is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate, etc., and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

Lentiviral transfer vectors Naldini et al. (1996), have been used to infect human cells growth-arrested in vitro and to transduce neurons after direct injection into the brain of adult rats. The vector was efficient at transferring marker genes in vivo into the neurons and long term expression in the absence of detectable pathology was achieved. Animals analyzed ten months after a single injection of the vector showed no decrease in the average level of transgene expression and no sign of tissue pathology or immune reaction (Blomer et al., 1997). Thus, in the present disclosure, one may graft or transplant cells infected with the recombinant lentivirus ex vivo, or infect cells in vivo.

Other Viral Vectors. The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present disclosure and may be selected according to the requisite properties of the target system.

In certain embodiments, vaccinia viral vectors are contemplated for use in the present disclosure. Vaccinia virus is a particularly useful eukaryotic viral vector system for expressing heterologous genes. For example, when recombinant vaccinia virus is properly engineered, the proteins are synthesized, processed and transported to the plasma membrane. Vaccinia viruses as gene delivery vectors have recently been demonstrated to transfer genes to human tumor cells, e.g., EMAP-II (Gnant et al., 1999), inner ear (Derby et al., 1999), glioma cells, e.g., p53 (Timiryasova et al., 1999) and various mammalian cells, e.g., P₄₅₀ (U.S. Pat. No. 5,506,138). The preparation, growth and manipulation of vaccinia viruses are described in U.S. Pat. Nos. 5,849,304 and 5,506,138 (each specifically incorporated herein by reference).

In other embodiments, sindbis viral vectors are contemplated for use in gene delivery. Sindbis virus is a species of the alphavirus genus (Garoff and Li, 1998) which includes such important pathogens as Venezuelan, Western and Eastern equine encephalitis viruses (Sawai et al., 1999; Mastrangelo et al., 1999). In vitro, sindbis virus infects a variety of avian, mammalian, reptilian, and amphibian cells. The genome of sindbis virus consists of a single molecule of single-stranded RNA, 11,703 nucleotides in length. The genomic RNA is infectious, is capped at the 5′ terminus and polyadenylated at the 3′ terminus, and serves as mRNA. Translation of a vaccinia virus 26S mRNA produces a polyprotein that is cleaved co- and post-translationally by a combination of viral and presumably host-encoded proteases to give the three virus structural proteins, a capsid protein (C) and the two envelope glycoproteins (E1 and PE2, precursors of the virion E2).

Three features of sindbis virus suggest that it would be a useful vector for the expression of heterologous genes. First, its wide host range, both in nature and in the laboratory. Second, gene expression occurs in the cytoplasm of the host cell and is rapid and efficient. Third, temperature-sensitive mutations in RNA synthesis are available that may be used to modulate the expression of heterologous coding sequences by simply shifting cultures to the non-permissive temperature at various time after infection. The growth and maintenance of sindbis virus is known in the art (U.S. Pat. No. 5,217,879, specifically incorporated herein by reference).

Chimeric Viral Vectors. Chimeric or hybrid viral vectors are being developed for use in therapeutic gene delivery and are contemplated for use in the present disclosure. Chimeric poxviral/retroviral vectors (Holzer et al., 1999), adenoviral/retroviral vectors (Feng et al., 1997; Bilbao et al., 1997; Caplen et al., 1999) and adenoviral/adeno-associated viral vectors (Fisher et al., 1996; U.S. Pat. No. 5,871,982) have been described.

These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

The adenovirus/AAV chimeric virus uses adenovirus nucleic acid sequences as a shuttle to deliver a recombinant AAV/transgene genome to a target cell. The adenovirus nucleic acid sequences employed in the hybrid vector can range from a minimum sequence amount, which requires the use of a helper virus to produce the hybrid virus particle, to only selected deletions of adenovirus genes, which deleted gene products can be supplied in the hybrid viral production process by a selected packaging cell. At a minimum, the adenovirus nucleic acid sequences employed in the pAdA shuttle vector are adenovirus genomic sequences from which all viral genes are deleted and which contain only those adenovirus sequences required for packaging adenoviral genomic DNA into a preformed capsid head. More specifically, the adenovirus sequences employed are the cis-acting 5′ and 3′ inverted terminal repeat (ITR) sequences of an adenovirus (which function as origins of replication) and the native 5′ packaging/enhancer domain, that contains sequences necessary for packaging linear Ad genomes and enhancer elements for the E1 promoter. The adenovirus sequences may be modified to contain desired deletions, substitutions, or mutations, provided that the desired function is not eliminated.

The AAV sequences useful in the above chimeric vector are the viral sequences from which the rep and cap polypeptide encoding sequences are deleted. More specifically, the AAV sequences employed are the cis-acting 5′ and 3′ inverted terminal repeat (ITR) sequences. These chimeras are characterized by high titer transgene delivery to a host cell and the ability to stably integrate the transgene into the host cell chromosome (U.S. Pat. No. 5,871,983, specifically incorporate herein by reference). In the hybrid vector construct, the AAV sequences are flanked by the selected adenovirus sequences discussed above. The 5′ and 3′ AAV ITR sequences themselves flank a selected transgene sequence and associated regulatory elements, described below. Thus, the sequence formed by the transgene and flanking 5′ and 3′ AAV sequences may be inserted at any deletion site in the adenovirus sequences of the vector. For example, the AAV sequences are desirably inserted at the site of the deleted E1a/E1b genes of the adenovirus. Alternatively, the AAV sequences may be inserted at an E3 deletion, E2a deletion, and so on. If only the adenovirus 5′ ITR/packaging sequences and 3′ ITR sequences are used in the hybrid virus, the AAV sequences are inserted between them.

The transgene sequence of the vector and recombinant virus can be a gene, a nucleic acid sequence or reverse transcript thereof, heterologous to the adenovirus sequence, which encodes a protein, polypeptide or peptide fragment of interest. The transgene is operatively linked to regulatory components in a manner which permits transgene transcription. The composition of the transgene sequence will depend upon the use to which the resulting hybrid vector will be put. For example, one type of transgene sequence includes a therapeutic gene which expresses a desired gene product in a host cell. These therapeutic genes or nucleic acid sequences typically encode products for administration and expression in a patient in vivo or ex vivo to replace or correct an inherited or non-inherited genetic defect or treat an epigenetic disorder or disease.

### 10. Non-Viral Transformation

Suitable methods for nucleic acid delivery for transformation of an organelle, a cell, a tissue or an organism for use with the current disclosure are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection. In certain embodiments, a nucleic acid may be delivered to an organelle, a cell, a tissue or an organism via one or more injections (i.e., a needle injection), such as, for example, either subcutaneously, intradermally, intramuscularly, intervenously or intraperitoneally. Methods of injection of vaccines are well known to those of ordinary skill in the art (e.g., injection of a composition comprising a saline solution). Further embodiments of the present disclosure include the introduction of a nucleic acid by direct microinjection. Direct microinjection has been used to introduce nucleic acid constructs into Xenopus oocytes (Harland and Weintraub, 1985).

Electroporation. In certain embodiments of the present disclosure, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human κ-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 92/17598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Calcium Phosphate. In other embodiments of the present disclosure, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran: In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading. Additional embodiments of the present disclosure include the introduction of a nucleic acid by direct sonic loading. LTK⁻ fibroblasts have been transfected with the thymidine kinase gene by sonication loading (Fechheimer et al., 1987).

Liposome-Mediated Transfection. In a further embodiment of the disclosure, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the disclosure, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor-Mediated Transfection. Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present disclosure.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present disclosure, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present disclosure can be specifically delivered into a target cell in a similar manner.

### 11. Expression Systems

Numerous expression systems exist that comprise at least a part or all of the compositions discussed above. Prokaryote- and/or eukaryote-based systems can be employed for use with the present disclosure to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

### E. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody construct of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody construct. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### F. Multispecific Antibody Construct Formats

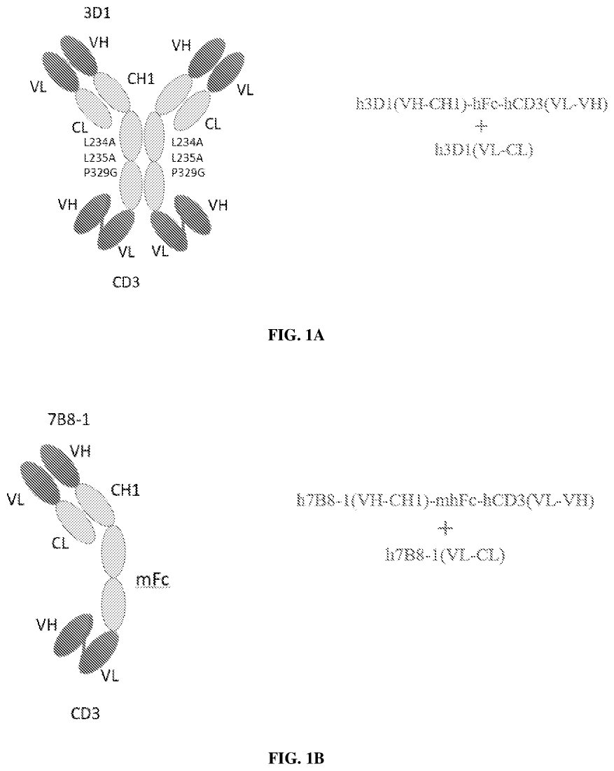

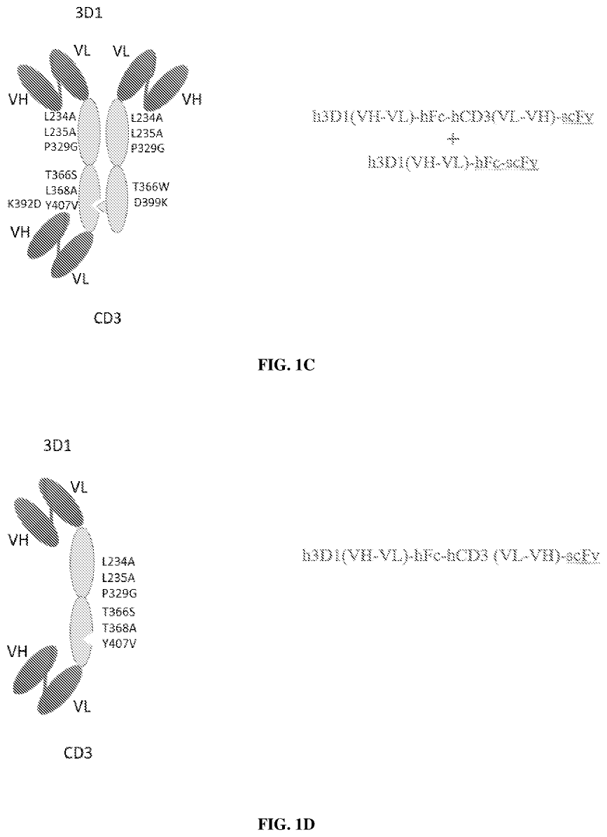

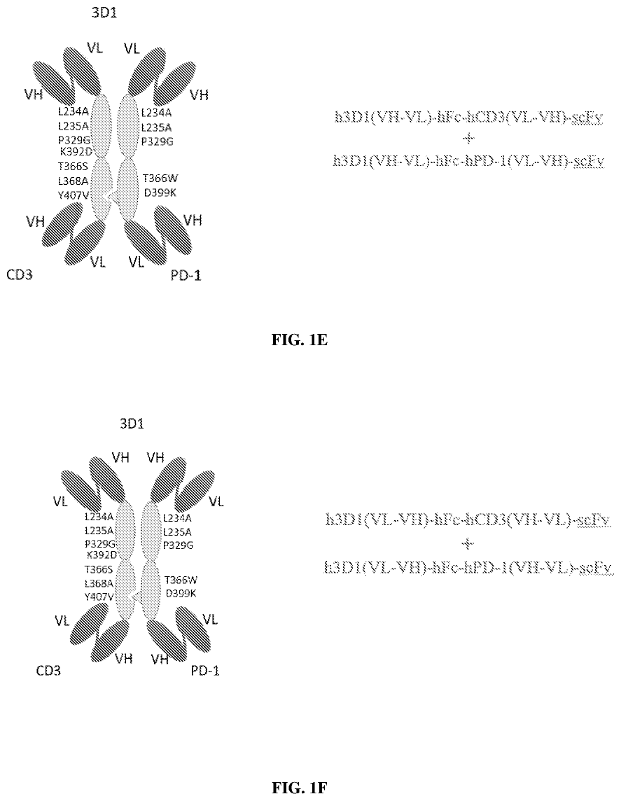

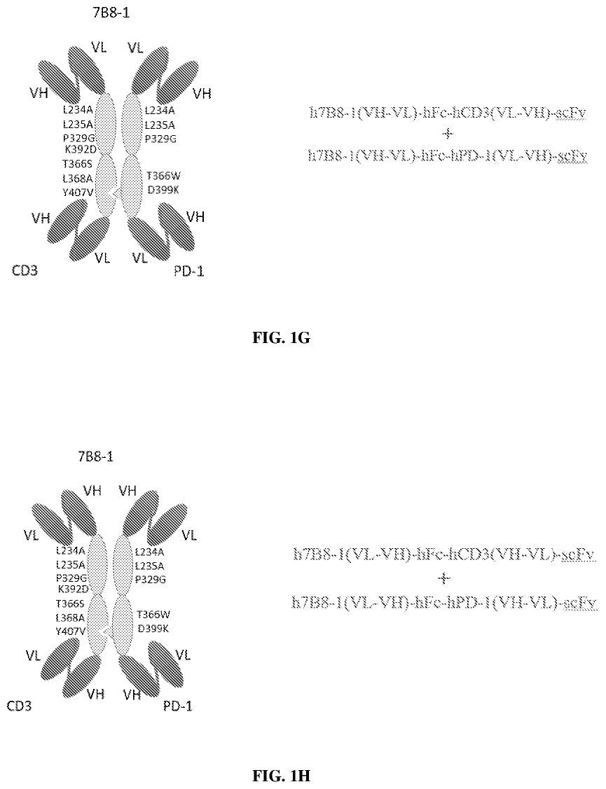

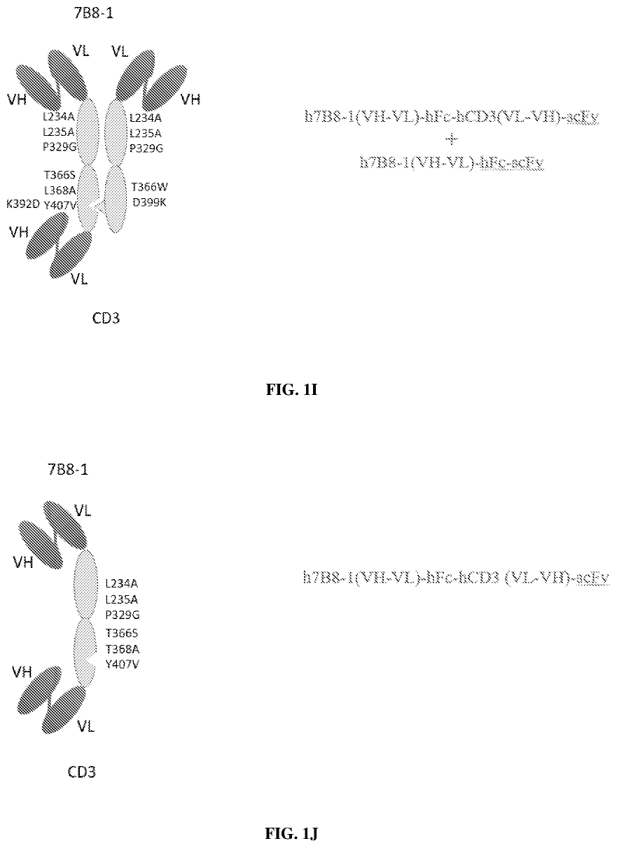

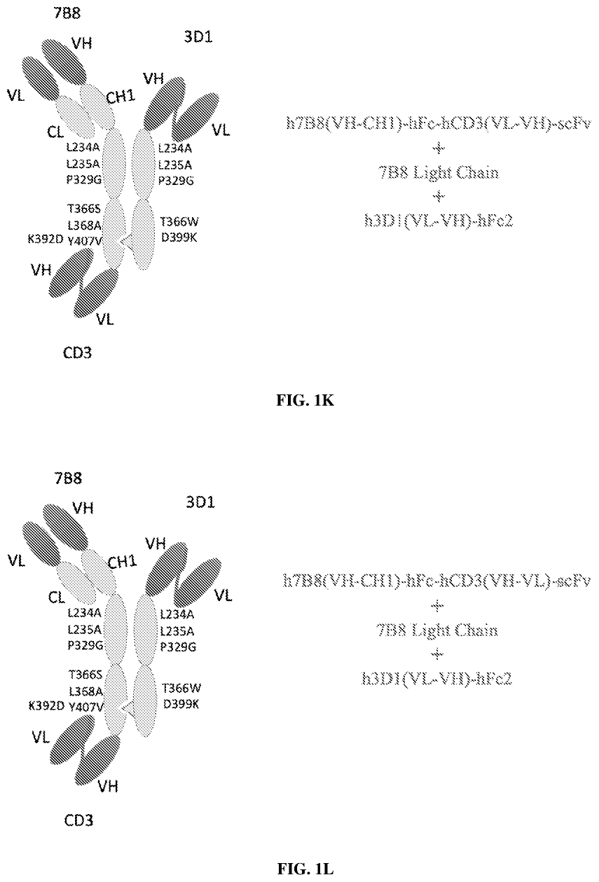

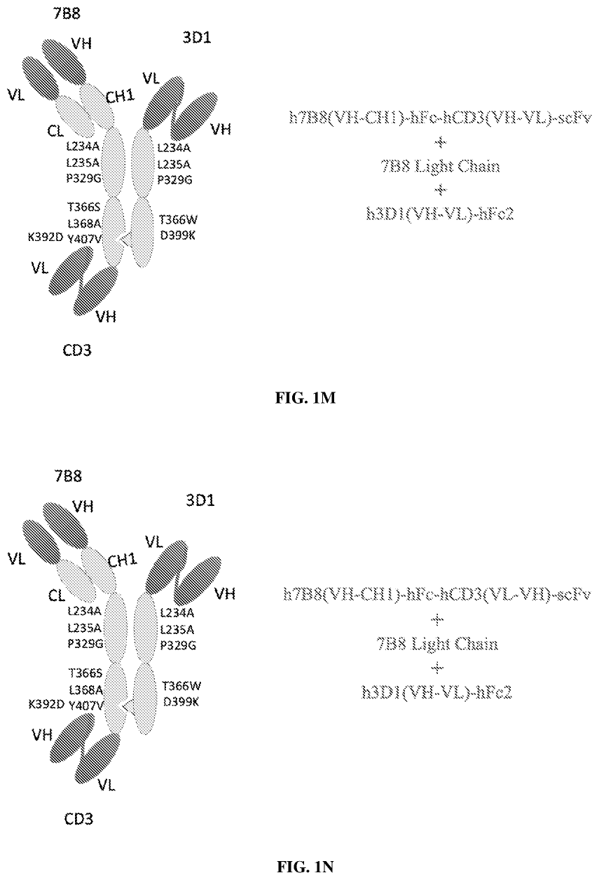

Multispecific antibodies are those that carry binding specificities for at least two different epitopes or antigens. The formats vary from what appears be to a traditional bivalent antibody with a different binding specificity grafted into on of the heavy/light chain variable region arms. Other formats use dual or triple single chain arrangments, some employing Fc component while others do not. Various formats are shown in FIG. 1A-J.

In addition to having one or two distinct binding specificities for MUC1-C, the multispecific antibodies of the present application may also bind one or two of the following antigens:

CD3. CD3 (cluster of differentiation 3) is a protein complex and T cell co-receptor that is involved in activating both the cytotoxic T cell (CD8+ naive T cells) and T helper cells (CD4+ naive T cells). It is composed of four distinct chains. In mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex.

The CD3γ, CD3δ, and CD3ε chains are highly related cell-surface proteins of the immunoglobulin superfamily containing a single extracellular immunoglobulin domain. A structure of the extracellular and transmembrane regions of the CD3γε/CD3δε/CD3ζζ/TCRαβ complex was solved with CryoEM, showing for the first time how the CD3 transmembrane regions enclose the TCR transmembrane regions in an open barrel. Containing aspartate residues, the transmembrane region of the CD3 chains is negatively charged, a characteristic that allows these chains to associate with the positively charged TCR chains. The intracellular tails of the CD3γ, CD3ε, and CD3δ molecules each contain a single conserved motif known as an immunoreceptor tyrosine-based activation motif or ITAM for short, which is essential for the signaling capacity of the TCR. The intracellular tail of CD3ζ contains 3 ITAM motifs.

Commercially available antibodies against CD3 include murmonab, oltelixizumab, teplizumab and visilizumab.

CD16. CD16, also known as FcγRIII, is a cluster of differentiation molecule found on the surface of natural killer cells, neutrophils, monocytes, and macrophages. CD16 has been identified as Fe receptors FcγRIIIa (CD16a) and FcγRIIIb (CD16b), which participate in signal transduction. The most well-researched membrane receptor implicated in triggering lysis by NK cells, CD16 is a molecule of the immunoglobulin superfamily (IgSF) involved in antibody-dependent cellular cytotoxicity (ADCC). It can be used to isolate populations of specific immune cells through fluorescent-activated cell sorting (FACS) or magnetic-activated cell sorting, using antibodies directed towards CD16.

CD16 is the type III Fcγ receptor. In humans, it exists in two different forms: FcγRIIIa (CD16a) and FcγRIIIb (CD16b), which have 96% sequence similarity in the extracellular immunoglobulin binding regions. While FcγRIIIa is expressed on mast cells, macrophages, and natural killer cells as a transmembrane receptor, FcγRIIIb is only expressed on neutrophils. In addition, FcγRIIIb is the only Fc receptor anchored to the cell membrane by a glycosyl-phosphatidylinositol (GPI) linker, and also plays a significant role in triggering calcium mobilization and neutrophil degranulation. FcγRIIIa and FcγRIIIb together are able to activate degranulation, phagocytosis, and oxidative burst, which allows neutrophils to clear opsonized pathogens.

Commercially available antibodies against CD28 are available from Novus Biologicals, Invitrogen-Thermo Fisher Scientific, Bio-Rad, Miltenyi Biotec, BD Biosciences and Agilent.

CD28. CD28 (Cluster of Differentiation 28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular).

CD28 is the receptor for CD80 (B7.1) and CD86 (B7.2) proteins. When activated by Toll-like receptor ligands, the CD80 expression is upregulated in antigen-presenting cells (APCs). The CD86 expression on antigen-presenting cells is constitutive (expression is independent of environmental factors). CD28 is the only B7 receptor constitutively expressed on naive T cells. Association of the TCR of a naive T cell with MHC:antigen complex without CD28:B7 interaction results in a T cell that is anergic.

CD28 possesses an intracellular domain with several residues that are critical for its effective signaling. The YMNM motif beginning at tyrosine 170 in particular is critical for the recruitment of SH2-domain containing proteins, especially PI3K, Grb2 and Gads. The Y170 residue is important for the induction of Bcl-xL via mTOR and enhancement of IL-2 transcription via PKCθ, but has no effect on proliferation and results a slight reduction in IL-2 production. The N172 residue (as part of the YMNM) is important for the binding of Grb2 and Gads and seems to be able to induce IL-2 mRNA stability but not NF-κB translocation. The induction of NF-κB seems to be much more dependent on the binding of Gads to both the YMNM and the two proline-rich motifs within the molecule. However, mutation of the final amino acid of the motif, M173, which is unable to bind PI3K but is able to bind Grb2 and Gads, gives little NF-κB or IL-2, suggesting that those Grb2 and Gads are unable to compensate for the loss of PI3K. IL-2 transcription appears to have two stages; a Y170-dependent, PI3K-dependent initial phase which allows transcription and a PI3K-independent second phase which is dependent on formation of an immune synapse, which results in enhancement of IL-2 mRNA stability. Both are required for full production of IL-2.

CD28 also contains two proline-rich motifs that are able to bind SH3-containing proteins. Itk and Tec are able to bind to the N-terminal of these two motifs which immediately succeeds the Y170 YMNM; Lck binds the C-terminal. Both Itk and Lck are able to phosphorylate the tyrosine residues which then allow binding of SH2 containing proteins to CD28. Binding of Tec to CD28 enhances IL-2 production, dependent on binding of its SH3 and PH domains to CD28 and PIP3 respectively. The C-terminal proline-rich motif in CD28 is important for bringing Lck and lipid rafts into the immune synapse via filamin-A. Mutation of the two prolines within the C-terminal motif results in reduced proliferation and IL-2 production but normal induction of Bcl-xL. Phosphorylation of a tyrosine within the PYAP motif (Y191 in the mature human CD28) forms a high affinity-binding site for the SH2 domain of the src kinase Lck which in turn binds to the serine kinase PKCO.

Commercially available antibodies against CD28 are available from Novus Biologicals, Invitrogen-Thermo Fisher Scientific, Bio-Rad, Miltenyi Biotec, BD Biosciences and Beckman Coulter.

Myeloid specific antigen. Myeloid specifica antigen CD33 or Siglec-3 (sialic acid binding Ig-like lectin 3, SIGLEC3, SIGLEC-3, gp67, p67) is a transmembrane receptor expressed on cells of myeloid lineage. It is usually considered myeloid-specific, but it can also be found on some lymphoid cells. It binds sialic acids, therefore is a member of the SIGLEC family of lectins. The extracellular portion of this receptor contains two immunoglobulin domains (one IgV and one IgC2 domain), placing CD33 within the immunoglobulin superfamily. The intracellular portion of CD33 contains immunoreceptor tyrosine-based inhibitory motifs (ITIMs) that are implicated in inhibition of cellular activity.

CD33 can be stimulated by any molecule with sialic acid residues such as glycoproteins or glycolipids. Upon binding, the immunoreceptor tyrosine-based inhibition motif (ITIM) of CD33, present on the cytosolic portion of the protein, is phosphorylated and acts as a docking site for Src homology 2 (SH2) domain-containing proteins like SHP phosphatases. This results in a cascade that inhibits phagocytosis in the cell.

CD33 is the target of gemtuzumab ozogamicin (trade name: Mylotarg®; Pfizer/Wyeth-Ayerst Laboratories), an antibody-drug conjugate for the treatment of patients with acute myeloid leukemia. The drug is a recombinant, humanized anti-CD33 monoclonal antibody (IgG4 K antibody hP67.6) covalently attached to the cytotoxic antitumor antibiotic calicheamicin (N-acetyl-γ-calicheamicin) via a bifunctional linker (4-(4-acetylphenoxy)butanoic acid). On Sep. 1, 2017, the FDA approved Pfizer's Mylotarg. Gemtuzumab ozogamicin was initially approved by the U.S. Food and Drug Administration in 2000. However, during post marketing clinical trials researchers noticed a greater number of deaths in the group of patients who received gemtuzumab ozogamicin compared with those receiving chemotherapy alone. Based on these results, Pfizer voluntarily withdrew gemtuzumab ozogamicin from the market in mid-2010 but was reintroduced to the market in 2017. CD33 is also the target in vadastuximab talirine (SGN-CD33A), a novel antibody-drug conjugate being developed by Seattle Genetics, utilizing this company's ADC technology.

Macrophage specific antigen. CD47 is a ubiquitous 50 kDa five-spanning membrane receptor that belongs to the immunoglobulin superfamily. This receptor, also known as integrin-associated protein, mediates cell-to-cell communication by ligation to transmembrane signal-regulatory proteins SIRPα and SIRPγ and interacts with integrins. CD47 is also implicated in cell-extracellular matrix interactions via ligation with thrombospondins. Furthermore, CD47 is involved in many and diverse cellular processes, including apoptosis, proliferation, adhesion and migration. It also plays a key role in many immune and cardiovascular responses. Thus, this multifaceted receptor might be a central actor in the tumour microenvironment. Solid tumours are composed of not only cancer cells that actively proliferate but also other cell types including immune cells and fibroblasts that make up the tumour microenvironment. Tumour cell proliferation is strongly sustained by continuous sprouting of new vessels, which also represents a gate for metastasis. Moreover, infiltration of inflammatory cells is observed in most neoplasms. Much evidence has accumulated indicating that infiltrating leukocytes promote cancer progression. Given its ubiquitous expression on all the different cell types that compose the tumour microenvironment, targeting CD47 could represent an original therapeutic strategy in the field of oncology.

A critical innate macrophage checkpoint is the CD47/Signal-regulatory protein alpha (SIRPα) pathway, a druggable target, which delivers an antiphagocytic signal to macrophages that in-hibits destruction of cancer cells overexpressing CD47 (Cluster of Differentiation 47). Tumors that overexpress CD47 include acute myeloid leukemia(AML), acute lymphoblastic leukemia, chronic lymphocytic leukemia, multiple myeloma, myelodysplastic syn-drome (MDS), diffuse large B-cell lymphoma (DLBCL), mantle cell lymphoma, and marginal cell lymphoma, as well as bladder, brain, breast, colon, esophageal, gastric, kidney, leiomyosarcoma, liver, lung, melanoma, ovarian, pancreatic, and prostate cancer. In addition to promoting macrophage-mediated phagocytosis, CD47 antagonism is associated with in-creased dendritic cell and natural killer cell cytotoxicity, which contributes to the heightened interest that CD47/SIRPα antagonism has generated.

Magrolimab is a monoclonal antibody against CD47 and macrophage checkpoint inhibitor that is designed to interfere with recognition of CD47 by the SIRPα receptor on macrophages, thus blocking the signal used by cancer cells to avoid being ingested by macrophages. Other antibodies against CD47 are commercially available from Abeam, Invitrogen-Thermo Fisher, R&D Systems, Bio-Rad and Biovision Inc.

SIRPα. Signal regulatory protein α (SIRPα) is a regulatory membrane glycoprotein from SIRP family expressed mainly by myeloid cells and also by stem cells or neurons. SIRPα acts as inhibitory receptor and interacts with a broadly expressed transmembrane protein CD47, also called the “don't eat me” signal. This interaction negatively controls effector function of innate immune cells such as host cell phagocytosis. SIRPα diffuses laterally on the macrophage membrane and accumulates at a phagocytic synapse to bind CD47 and signal ‘self’, which inhibits the cytoskeleton-intensive process of phagocytosis by the macrophage. This is analogous to the self signals provided by MHC class I molecules to NK cells via Ig-like or Ly49 receptors. Protein shown to the right is CD47 not SIRP α.

The cytoplasmic region of SIRPα is highly conserved between rats, mice and humans. Cytoplasmic region contains a number of tyrosine residues, which likely act as ITIMs. Upon CD47 ligation, SIRPα is phosphorylated and recruits phosphatases like SHP1 and SHP2. The extracellular region contains three Immunoglobulin superfamily domains—single V-set and two C1-set IgSF domains. SIRP β and γ have the similar extracellular structure but different cytoplasmic regions giving contrasting types of signals. SIRP α polymorphisms are found in ligand-binding IgSF V-set domain but it does not affect ligand binding. One idea is that the polymorphism is important to protect the receptor of pathogens binding. SIRPα recognizes CD47, an anti-phagocytic signal that distinguishes live cells from dying cells. CD47 has a single Ig-like extracellular domain and five membrane spanning regions. The interaction between SIRPα and CD47 can be modified by endocytosis or cleavage of the receptor, or interaction with surfactant proteins. Surfactant protein A and D are soluble ligands, highly expressed in the lungs, that bind to the same region of SIRPα as CD47 and can therefore competitively block binding.

The extracellular domain of SIRP α binds to CD47 and transmits intracellular signals through its cytoplasmic domain. CD47-binding is mediated through the NH2-terminal V-like domain of SIRP α. The cytoplasmic region contains four ITIMs that become phosphorylated after binding of ligand. The phosphorylation mediates activation of tyrosine kinase SHP2. SIRP α has been shown to bind also phosphatase SHP1, adaptor protein SCAP2 and FYN-binding protein. Recruitment of SHP phosphatases to the membrane leads to the inhibition of myosin accumulation at the cell surface and results in the inhibition of phagocytosis.

Cancer cells highly expressed CD47 that activate SIRP α and inhibit macrophage-mediated destruction. In one study, they engineered high-affinity variants of SIRP α that antagonized CD47 on cancer cells and caused increase phagocytosis of cancer cells. Another study (in mice) found anti-SIRPα antibodies helped macrophages to reduce cancer growth and metastasis, alone and in synergy with other cancer treatments.

There are numerous commercially available anti-SIRPα antibodies from companies like Bio X Cell, Biolegend, Sino Biological, Thermo-Fisher, R&D Systems, and Arigo Bio.

erbB2. Receptor tyrosine-protein kinase erbB-2, also known as CD340 (cluster of differentiation 340), proto-oncogene Neu, Erbb2 (rodent), or ERBB2 (human), is a protein that in humans is encoded by the ERBB2 gene. ERBB is abbreviated from erythroblastic oncogene B, a gene isolated from avian genome. It is also frequently called HER2 (from human epidermal growth factor receptor 2) or HER2/neu.

HER2 is a member of the human epidermal growth factor receptor (HER/EGFR/ERBB) family. Amplification or over-expression of this oncogene has been shown to play an important role in the development and progression of certain aggressive types of breast cancer. In recent years the protein has become an important biomarker and target of therapy for approximately 30% of breast cancer patients.

HER2 is so named because it has a similar structure to human epidermal growth factor receptor, or HER1. Neu is so named because it was derived from a rodent glioblastoma cell line, a type of neural tumor. ErbB-2 was named for its similarity to ErbB (avian erythroblastosis oncogene B), the oncogene later found to code for EGFR. Molecular cloning of the gene showed that HER2, Neu, and ErbB-2 are all encoded by the same orthologs.

The erbB family consists of four plasma membrane-bound receptor tyrosine kinases. One of which is erbB-2, and the other members being epidermal growth factor receptor, erbB-3 (neuregulin-binding; lacks kinase domain), and erbB-4. All four contain an extracellular ligand binding domain, a transmembrane domain, and an intracellular domain that can interact with a multitude of signaling molecules and exhibit both ligand-dependent and ligand-independent activity. Notably, no ligands for HER2 have yet been identified. HER2 can heterodimerise with any of the other three receptors and is considered to be the preferred dimerisation partner of the other ErbB receptors. Dimerisation results in the autophosphorylation of tyrosine residues within the cytoplasmic domain of the receptors and initiates a variety of signaling pathways.

There are commercially available antibodies against erbB2 including trastuzumab, pertuzumab, and margetuximab.

EGFR. The epidermal growth factor receptor (EGFR; ErbB-1; HER1 in humans) is a transmembrane protein that is a receptor for members of the epidermal growth factor family (EGF family) of extracellular protein ligands. The epidermal growth factor receptor is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). In many cancer types, mutations affecting EGFR expression or activity could result in cancer. Deficient signaling of the EGFR and other receptor tyrosine kinases in humans is associated with diseases such as Alzheimer's, while over-expression is associated with the development of a wide variety of tumors. Interruption of EGFR signalling, either by blocking EGFR binding sites on the extracellular domain of the receptor or by inhibiting intracellular tyrosine kinase activity, can prevent the growth of EGFR-expressing tumours and improve the patient's condition.

EGFR is a transmembrane protein that is activated by binding of its specific ligands, including epidermal growth factor and transforming growth factor α (TGFα) ErbB2 has no known direct activating ligand, and may be in an activated state constitutively or become active upon heterodimerization with other family members such as EGFR. Upon activation by its growth factor ligands, EGFR undergoes a transition from an inactive monomeric form to an active homodimer—although there is some evidence that preformed inactive dimers may also exist before ligand binding. In addition to forming homodimers after ligand binding, EGFR may pair with another member of the ErbB receptor family, such as ErbB2/Her2/neu, to create an activated heterodimer. There is also evidence to suggest that clusters of activated EGFRs form, although it remains unclear whether this clustering is important for activation itself or occurs subsequent to activation of individual dimers.

EGFR dimerization stimulates its intrinsic intracellular protein-tyrosine kinase activity. As a result, autophosphorylation of several tyrosine (Y) residues in the C-terminal domain of EGFR occurs. These include Y992, Y1045, Y1068, Y1148 and Y1173, as shown in the adjacent diagram. This autophosphorylation elicits downstream activation and signaling by several other proteins that associate with the phosphorylated tyrosines through their own phosphotyrosine-binding SH2 domains. These downstream signaling proteins initiate several signal transduction cascades, principally the MAPK, Akt and JNK pathways, leading to DNA synthesis and cell proliferation. Such proteins modulate phenotypes such as cell migration, adhesion, and proliferation. Activation of the receptor is important for the innate immune response in human skin. The kinase domain of EGFR can also cross-phosphorylate tyrosine residues of other receptors it is aggregated with and can itself be activated in that manner.

There are commercially available antibodies against EGRF including cetuximab, panitumumab, nimotuzumab and necitumumab.

PD1. Programmed cell death protein 1, also known as PD1 and CD279 (cluster of differentiation 279), is a protein on the surface of cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells. PD-1 is an immune checkpoint and guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells). PD-1 inhibitors, a new class of drugs that block PD-1, activate the immune system to attack tumors and are used to treat certain types of cancer.

The PD-1 protein in humans is encoded by the PDCD1 gene. PD-1 is a cell surface receptor that belongs to the immunoglobulin superfamily and is expressed on T cells and pro-B cells. PD-1 binds two ligands, PD-L1 and PD-L2. PD-1 is a type I membrane protein of 288 amino acids. PD-1 is a member of the extended CD28/CTLA-4 family of T cell regulators. The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates T-cell receptor TCR signals. This is consistent with binding of SHP-1 and SHP-2 phosphatases to the cytoplasmic tail of PD-1 upon ligand binding. In addition, PD-1 ligation up-regulates E3-ubiquitin ligases CBL-b and c-CBL that trigger T cell receptor down-modulation. PD-1 is expressed on the surface of activated T cells, B cells, and macrophages, suggesting that compared to CTLA-4, PD-1 more broadly negatively regulates immune responses.

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines.

Several lines of evidence suggest that PD-1 and its ligands negatively regulate immune responses. PD-1 knockout mice have been shown to develop lupus-like glomerulonephritis and dilated cardiomyopathy on the C57BL/6 and BALB/c backgrounds, respectively. In vitro, treatment of anti-CD3 stimulated T cells with PD-L1-Ig results in reduced T cell proliferation and IFN-γ secretion. IFN-γ is a key pro-inflammatory cytokine that promotes T cell inflammatory activity. Reduced T cell proliferation was also correlated with attenuated IL-2 secretion and together, these data suggest that PD-1 negatively regulates T cell responses.

Experiments using PD-L1 transfected DCs and PD-1 expressing transgenic (Tg) CD4⁺ and CD8⁺ T cells suggest that CD8⁺ T cells are more susceptible to inhibition by PD-L1, although this could be dependent on the strength of TCR signaling. Consistent with a role in negatively regulating CD8⁺ T cell responses, using an LCMV viral vector model of chronic infection, Rafi Ahmed's group showed that the PD-1-PD-L1 interaction inhibits activation, expansion and acquisition of effector functions of virus specific CD8⁺ T cells, which can be reversed by blocking the PD-1-PD-L1 interaction.

Expression of PD-L1 on tumor cells inhibits anti-tumor activity through engagement of PD-1 on effector T cells. Expression of PD-L1 on tumors is correlated with reduced survival in esophageal, pancreatic and other types of cancers, highlighting this pathway as a target for immunotherapy. Triggering PD-1, expressed on monocytes and up-regulated upon monocytes activation, by its ligand PD-L1 induces IL-10 production which inhibits CD4 T-cell function.

In mice, expression of this gene is induced in the thymus when anti-CD3 antibodies are injected and large numbers of thymocytes undergo apoptosis. Mice deficient for this gene bred on a BALB/c background developed dilated cardiomyopathy and died from congestive heart failure. These studies suggest that this gene product may also be important in T cell function and contribute to the prevention of autoimmune diseases.

There are many commercially available antibodies against PD1 includes pemrolizumab, nivolumab, cemiplimab, atezolizumab, duravalumab and avelumab.

NKG2D. NKG2D is a transmembrane protein belonging to the NKG2 family of C-type lectin-like receptors. NKG2D is encoded by KLRK1 gene which is located in the NK-gene complex (NKC) situated on chromosome 6 in mice and chromosome 12 in humans. In mice, it is expressed by NK cells, NK1.1⁺ T cells, γδ T cells, activated CD8⁺ αβ T cells and activated macrophages. In humans, it is expressed by NK cells, γδ T cells and CD8⁺ αβ T cells. NKG2D recognizes induced-self proteins from MIC and RAET1/ULBP families which appear on the surface of stressed, malignant transformed, and infected cells.

Human NKG2D receptor complex assembles into a hexameric structure. NKG2D itself forms a homodimer whose ectodomains serve for ligand binding. Each NKG2D monomer is associated with DAP10 dimer. This association is maintained by ionic interaction of a positively charged arginine present in a transmembrane segment of NKG2D and negatively charged aspartic acids within both transmembrane regions of DAP10 dimer. DAP10 functions as an adaptor protein and transduces the signal after the ligand binding by recruiting the p85 subunit of PI3K and Grb2-Vav1 complex which are responsible for subsequent downstream events.

In mice, alternative splicing generates two distinct NKG2D isoforms: the long one (NKG2D-L) and the short one (NKG2D-S). NKG2D-L binds DAP10 similarly to human NKG2D. By contrast, NKG2D-S associates with two adaptor proteins: DAP10 and DAP12. DAP10 recruits the p85 subunit of PI3K and a complex of Grb2 and Vav1. DAP12 bears ITAM motif and activates protein tyrosine kinases Syk and Zap70 signalling.

NKG2D is a major recognition receptor for the detection and elimination of transformed and infected cells as its ligands are induced during cellular stress, either as a result of infection or genomic stress such as in cancer. In NK cells, NKG2D serves as an activating receptor, which itself is able to trigger cytotoxicity. The function of NKG2D on CD8⁺ T cells is to send co-stimulatory signals to activate them.

NKG2D ligands are induced-self proteins which are completely absent or present only at low levels on surface of normal cells, but they are overexpressed by infected, transformed, senescent and stressed cells. Their expression is regulated at different stages (transcription, mRNA and protein stabilization, cleavage from the cell surface) by various stress pathways. Among them, one of the most prominent stress pathways is DNA damage response. Genotoxic stress, stalled DNA replication, poorly regulated cell proliferation in tumorigenesis, viral replication or some viral products activate the ATM and ATR kinases. These kinases initiate the DNA damage response pathway which participates in NKG2D ligand upregulation. DNA damage response thus participate in alerting the immune system to the presence of potentially dangerous cells.

All NKG2D ligands are homologous to MHC class I molecules and are divided into two families: MIC and RAET1/ULBP. Commercially available antibodies against NKG2D are available from Invitrogen, Abcam, BioLegend, Bio X Cell, R&D Systems, EMD Millipore and Milteny Biotec.

Siglec-9. Due to the aberrant glycosylation present in cancer, the multiple O-linked glycans carried by MUC1 are mainly short and sialylated, in contrast to the long, branched chains seen on MUC1 expressed by normal epithelial cells. In carcinomas, the aberrant 0-linked glycosylation of MUC1 can alter the interaction of MUC1 with lectins of the immune system and can thereby influence tumor-immune system interplay. Siglec-9 is expressed predominantly on myeloid cells. Siglecs (‘sialic-acid-binding immunoglobulin-like lectins’) are a family of sialic-acid-binding lectins that are expressed on various cells of the immune system. The cytoplasmic domains of most Siglecs contain immunoreceptor tyrosine-based inhibitory motifs (ITIMs) that recruit the tyrosine phosphatases SHP-1 and SHP-2 and thus regulate the cells of the innate and adaptive immune responses. It has become clear that Siglecs have a role in cancer immunosuppression, as the hyper-sialylation seen in cancers induces binding to these lectins

### III. PHARMACEUTICAL FORMULATIONS AND TREATMENT OF CANCER

### A. Cancers

Cancer results from the outgrowth of a clonal population of cells from tissue. The development of cancer, referred to as carcinogenesis, can be modeled and characterized in a number of ways. An association between the development of cancer and inflammation has long-been appreciated. The inflammatory response is involved in the host defense against microbial infection, and also drives tissue repair and regeneration. Considerable evidence points to a connection between inflammation and a risk of developing cancer, i.e., chronic inflammation can lead to dysplasia.

Cancer cells to which the methods of the present disclosure can be applied include generally any cell that expresses MUC1, and more particularly, that overexpresses MUC1. An appropriate cancer cell can be a breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, or bladder cancer cell. In addition, the methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice. Cancers may also be recurrent, metastatic and/or multi-drug resistant, and the methods of the present disclosure may be particularly applied to such cancers so as to render them resectable, to prolong or re-induce remission, to inhibit angiogenesis, to prevent or limit metastasis, and/or to treat multi-drug resistant cancers. At a cellular level, this may translate into killing cancer cells, inhibiting cancer cell growth, or otherwise reversing or reducing the malignant phenotype of tumor cells.

### B. Formulation and Administration

The present disclosure provides pharmaceutical compositions comprising anti-MUC1-C antibody constructs. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Other suitable pharmaceutical excipients include starch, glucose, lactose, sucrose, saline, dextrose, gelatin, malt, rice, flour, chalk, silica gel, sodium stearate, glycerol monostearate, talc, sodium chloride, dried skim milk, glycerol, propylene glycol, water, ethanol and the like.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The antibodies of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or administration local or regional to a tumor, for example, in the local or regional vasculature or lymphatic system, or in a resected tumor bed.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

### C. Combination Therapies

In the context of the present disclosure, it also is contemplated that anti-MUC1-C antibody constructs described herein could be used similarly in conjunction with chemo- or radiotherapeutic intervention, or other treatments. It also may prove effective, in particular, to combine anti-MUC1-C/ECD antibodies with other therapies that target different aspects of MUC1 function, such as peptides and small molecules that target the MUC1 cytoplasmic domain.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with an anti-MUC1-C antibody construct according to the present disclosure and at least one other agent. These compositions would be provided in a combined amount effective to kill or inhibit proliferation of the cell. This process may involve contacting the cells with the anti-MUC1-C antibody construct according to the present disclosure and the other agent(s) or factor(s) at the same time. This may be achieved by contacting the cell with a single composition or pharmacological formulation that includes both agents, or by contacting the cell with two distinct compositions or formulations, at the same time, wherein one composition includes the anti-MUC1-C antibody construct according to the present disclosure and the other includes the other agent.

Alternatively, the anti-MUC1-C antibody construct therapy may precede or follow the other agent treatment by intervals ranging from minutes to weeks. In embodiments where the other agent and the anti-MUC1 antibody construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either anti-MUC1 antibody construct or the other agent will be desired. Various combinations may be employed, where an anti-MUC1-C antibody construct according to the present disclosure therapy is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell killing, both agents are delivered to a cell in a combined amount effective to kill the cell.

Agents or factors suitable for cancer therapy include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” may be used. This may be achieved by irradiating the localized tumor site; alternatively, the tumor cells may be contacted with the agent by administering to the subject a therapeutically effective amount of a pharmaceutical composition.

Various classes of chemotherapeutic agents are comtemplated for use with the present disclosure. For example, selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene.

Chemotherapeutic agents contemplated to be of use, include, e.g., camptothecin, actinomycin-D, mitomycin C. The disclosure also encompasses the use of a combination of one or more DNA damaging agents, whether radiation-based or actual compounds, such as the use of X-rays with cisplatin or the use of cisplatin with etoposide. The agent may be prepared and used as a combined therapeutic composition, or kit, by combining it with a MUC1 peptide, as described above.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for doxorubicin, to 35-50 mg/m² for etoposide intravenously or double the intravenous dose orally. Microtubule inhibitors, such as taxanes, also are contemplated. These molecules are diterpenes produced by the plants of the genus Taxus, and include paclitaxel and docetaxel.

Epidermal growth factor receptor inhibitors, such as Iressa, mTOR, the mammalian target of rapamycin, also known as FK506-binding protein 12-rapamycin associated protein 1 (FRAP1) is a serine/threonine protein kinase that regulates cell growth, cell proliferation, cell motility, cell survival, protein synthesis, and transcription. Rapamycin and analogs thereof (“rapalogs”) are therefore contemplated for use in cancer therapy in accordance with the present disclosure.

Another possible therapy is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, x-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

A particular mode for delivery of radiotherapeutics is nanoparticles. For example, gold nanoparticles (NPs) were the first NP-based radio-enhancers to be tested in small animals for tumor therapy. Their ability to augment the efficacy of external bean radiation was found to be mediated via the photo-electric effect and by Auger electron showers that arise due to the interactions between gold atoms and low energy photons produced by the external beam. Based on these early findings, various inorganic NPs have been developed to similarly boost the efficacy of radiation therapy, including ones composed of bismuth, hafnium, and gadolinium, among others. Various approaches have been adopted to improve the internalization of radioenhancers in preclinical tumor models, including through functionalization of NPs with antibodies, to aid in tumor targeting. Efforts have also focused on optimization of the timing of radiation via imaging of the same NP construct by computed tomography (CT) or by magnetic resonance imaging.

Hafnium oxide-based NPs (NBTXR3) injected intra-tumorally in a hydrogel have been imaged effectively by CT, demonstrating persistence inside the tumor bed post-implantation as well as limited diffusion outside of the injection site. In parallel, gadolinium-containing NPs (AGuIX) that were administered intravenously (IV) have been successfully tracked by magnetic resonance imaging, enabling radiation therapy only after tumor localiza-tion. Both approaches are promising and support the generalized capability of inorganic NPs to serve as radioenhancers in clinical applications. Although IV injection of imaging agents enables accessibility to a multitude of cancers, NPs administered via the IV route have been shown to rapidly wash out from tumors if not internalized by tumor cells as was observed in the NANO-RAD trial (NCT02820454).

In a recent study, Detappe et al. (2020) hypothesized that NPs that are engineered to persist within the tumor environment could more effectively enhance the dose of fractionated radiation treatment and could obviate the need for repeated radio-enhancer administration, which could decrease potential morbidities and/or treatment-related costs. The authors conjugated multiple NPs to a single tumor-specific monoclonal antibody (mAb) to increase the dose of radioenhancer that is delivered to tumor cells. As the target for these antibody-conjugated NPs, they selected mucin 1 (MUC1) based on its high expression levels across a variety of solid and hematologic malignancies. To compare the radioenhancement properties of MUC1-Cantibody-conjugated NPs to their unconjugated counterparts, the authors used the same type of nanoparticles that were used in the NANO-RAD trial and both compositions were administered in combination with either a single high dose of external beam or with fractionated radiation therapy, and treatment effects were compared in various models of lung and triple-negative breast cancer. The % ID/g of anti-MUC1-C/NPs that accumulated within tumors was found to be similar to that of their unconjugated counterparts. Importantly, the anti-MUC1-C/NPs demonstrated prolonged retention in in vivo tumor microenvironments; as a result, the radiation boost was maintained during the course of fractionated therapy (3×5.2 Gy). The authors found that by administering anti-MUC1-C/NPs with XRT, it was possible to significantly augment tumor growth inhibition and to prolong the animals' overall survival (46.2±3.1 days) compared with the administration of control NPs with XRT (31.1+2.4 days) or with XRT alone (27.3±1.6 days; P<0.01, log-rank).

In addition, it also is contemplated that immunotherapy, hormone therapy, toxin therapy and surgery can be used. In particular, one may employ targeted therapies such as Avastin, Erbitux, Gleevec, Herceptin and Rituxan.

One particularly advantageous approach to combination therapy is to select a second agent that targets MUC1. In copending application filed by the present inventors, there are disclosed methods of inhibiting a MUC1-positive tumor cell in a subject comprising administering to said subject a MUC1 peptide of at least 4 consecutive MUC1 residues and no more than 20 consecutive MUC1 residues and comprising the sequence CQC, wherein the amino-terminal cysteine of CQC is covered on its NH₂-terminus by at least one amino acid residue that need not correspond to the native MUC-1 transmembrane sequence. The peptide may comprise at least 5 consecutive MUC1 residues, at least 6 consecutive MUC1 residues, at least 7 consecutive MUC1 residues, at least 8 consecutive MUC1 residues and the sequence may more specifically comprise CQCR (SEQ ID NO: 15), CQCRR (SEQ ID NO: 16), CQCRRR (SEQ ID NO: 17), CQCRRRR (SEQ ID NO: 18), CQCRRK (SEQ ID NO: 19), CQCRRKN (SEQ ID NO: 20), or RRRRRRRRRCQCRRKN (SEQ ID NO: 21). The peptide may contain no more than 10 consecutive residues, 11 consecutive residues, 12 consecutive residues, 13 consecutive residues, 14 consecutive residues, 15 consecutive residues, 16 consecutive residues, 17 consecutive residues, 18 consecutive residues or 19 consecutive residues of MUC1. The peptide may be fused to a cell delivery domain, such as poly-D-R, poly-D-P or poly-D-K. The peptide may comprise all L amino acids, all D amino acids, or a mix of L and D amino acids. See U.S. Pat. No. 8,524,669.

A variation on this technology is described in U.S. patent application Ser. No. 13/026,858. In that application, methods of inhibiting a MUC1-positive cancer cell are disclosed comprising contacting the cell with a MUC1 peptide of at least 4 consecutive MUC1 residues and no more than 20 consecutive MUC1 residues and comprising the sequence CQC, wherein (i) the amino-terminal cysteine of CQC is covered on its NH₂-terminus by at least one amino acid residue that need not correspond to the native MUC1 transmembrane sequence; and (ii) the peptide comprises 3-5 consecutive positively-charged amino acid residues in addition to those positively-charged amino acid residues corresponding to native MUC1 residues. The MUC1-positive cell may be a solid tumor cell, such as a lung cancer cell, a brain cancer cell, a head & neck cancer cell, a breast cancer cell, a skin cancer cell, a liver cancer cell, a pancreatic cancer cell, a stomach cancer cell, a colon cancer cell, a rectal cancer cell, a uterine cancer cell, a cervical cancer cell, an ovarian cancer cell, a testicular cancer cell, a skin cancer cell or a esophageal cancer cell. The MUC1-positive cell may be a leukemia or myeloma cell, such as acute myeloid leukemia, chronic myelogenous leukemia or multiple myeloma. The peptide may be a stapled peptide, a cyclized peptide, a peptidomimetic or peptoid. The method may further comprise contacting the cell with a second anti-cancer agent, such as where the second anti-cancer agent is contacted prior to the peptide, after the peptide or at the same time as the peptide. Inhibiting may comprise inhibiting cancer cell growth, cancer cell proliferation or inducing cancer cell death, such as by apoptosis.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### IV. KITS

In still further embodiments, there are kits for use with the methods described herein. The kits will thus comprise, in suitable container means, an antibody construct as described here. The components of the kits may be packaged either in aqueous media or in lyophilized form. The kits may also include instructions for use of the antibody constructs.

The container means of the kits will generally include at least one vial, test tube, flask, bottle, syringe or other container means, into which the antibody construct may be placed, or preferably, suitably aliquoted. The kits will also include a means for containing the antibody, antigen, and any other reagent containers in close confinement for commercial sale. Such containers may include injection or blow-molded plastic containers into which the desired vials are retained.

### V. EXAMPLES

The following examples are included to demonstrate preferred embodiments. It should be appreciated by those of skill in the art that the techniques disclosed in the examples which follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

h3D1-hCD3 bispecific antibody with separate light chain. h3D1-hCD3 is a homodimer that contains bivalent h3D1 and bivalent hCD3 binding paratopes along with LALA-PG mutation to abolish any Fc receptor mediated effector mechanism. A construct containing the scFv format was generated by fusing various domains of two different antibodies in the following order: The N-terminal of the ScFv contains the VH domain of 3D1 antibody along with CH1 domain followed by Fc region of human IgG1. The C-terminal of the Fc was fused to the VL and VH domains of the CD3 antibody via a glycine-serine linker that will enable the flexibility and folding of the individual domains. A second construct containing the VL and CL regions of h3D1 also was generated. When these two constructs are co-expressed in CHO cells, it forces a canonical immunoglobulin structure to be formed by pairing the light chain of h3D1 with the heavy chain of h3D1 and also a homodimer of the protein is forced to form through disulfide linkers in the Fc region like a native immunoglobulin molecule. The human IgG1 Fc includes three mutations (L234A, L235A, P329G) that abrogates the binding to the Fc receptors of hematopoietic cells and C1q, a component of the complement system and thereby it can minimize the secondary immunological reactions such cytokine release syndrome and complement activation. See FIG. 1A.

h7B8-1-hCD3 bispecific antibody. h7B8-1-hCD3 is a monomer containing a separate light chain. The affinity of humanized 7B8-1 (h7B8-1) is 10 times more than humanized 3D1. Therefore, a h7B8-1-hCD3 bispecific construct was generated to have a single MUC1 binding site by incorporating a monomeric Fc that has better stability and does not dimerize. A construct was generated by fusing different domains of 7B8-1 and anti-CD3 antibodies in the following order: The N-terminal of the construct contains the VH domain of 7B8-1 antibody along with CH1 domain followed by monomeric human Fc region. The C-terminal of the monomeric human Fc was fused to the VL and VH domains of the CD3 antibody via a glycine-serine linker that will enable the flexibility and folding of the individual domains. A second construct containing VL and VH domains of the h3D1 antibody was also expressed that can pair with the VH of h3D1. See FIG. 1B.

h3D1-hCD3 bispecific antibody. h3D1-hCD3 is a heterodimer with scFvs brought together via knob-into-hole binding. This construct has bivalent binding site for MUC1 and monovalent binding site for CD3 due to heterodimerization by using knobs-into-hole technology with the indicated mutations (T366S, T368A, Y407V against T366W) in the Fc region. The knobs-into-hole technology applies large amino acids in one chain to create a “knob” and employs smaller amino acids for a corresponding “hole” in the other chain. In addition, electrostatic steering of two oppositely charged heavy chains in combination with the single chain variable fragment (scFv) technology ensures correct chain assembly. A construct was generated by fusing different domains humanized 3D1 and humanized CD3 antibodies in the following order: The N-terminal of the construct contains the VH domain of h3D1 antibody was fused with VL domain using a glycine-serine linker followed by Fc region of human IgG1. The C-terminal of the Fc was fused to the VL and VH domains of the CD3 antibody via a glycine-serine linker that will enable the flexibility and folding of the individual domains. The Fc region contains three mutations (T366S, T368A, Y407V) that form the hole and another mutation (K392D) that creates an electrostatic steering for proper chain pairing. A second construct containing the VH domain fused to VL domain via a glycine-serine linker followed by Fc region of hIgG1 with a mutation that form the knob was generated (T366W). The human IgG1 Fc includes three mutations (L234A, L235A, P329G) that abrogates the binding to the Fc receptors of hematopoietic cells and C1q, a component of the complement system and thereby it can minimize the secondary immunological reactions such cytokine release syndrome and complement activation. See FIG. 1C.

h3D1-hCD3 bispecific antibody (scFv). This format of bispecific antibody has a single chain variable fragment (scFv) that has one binding site each for MUC1 and CD3 and remains as a monomer due to the indicated mutations. A construct was generated by fusing the VL domain of h3D1 with the Fc of human IgG1 followed by adding the VL domain of humanized CD3 antibody. VH domain of h3D1 was added to the N-terminal and VH domain of humanized CD3 antibody was added to the C-terminal of the construct using glycine-serine linker on both ends. The Fc of human IgG1 contains mutations (L234A, L235A, P329G) to abrogate the Fc receptor mediated effector mechanism as well as C1q binding. See FIG. 1D.

h3D1-hCD3-hPD1 tri-specific antibody. This Dual Immune Cell Engager (DICE) format employs the same strategy of heterodimerization as in FIG. 1C, but it includes a binding site for PD1 at the N-terminal of the second construct. Addition of a PD-1 binding site enhances T-cell activation by blocking the checkpoint inhibition caused by PD-1 and PD-L1 interaction. See FIG. 1E.

h3D1-hCD3-hPD1 tri-specific antibody. h3D1-hCD3-hPD1 is a heterodimer with scFvs brought together via knob-into-hole binding. This construct has bivalent binding site for MUC1 and monovalent binding site for CD3 and monovalent binding site for PD1. Heterodimerization by using knobs-into-hole technology with the indicated mutations (T366S, T368A, Y407V against T366W) in the Fc region. A construct was generated by fusing different domains humanized h3D1 and humanized CD3 and PD1 antibodies in the following order: The N-terminal of the construct contains the VL domain of h3D1 antibody was fused with VH domain using a glycine-serine linker followed by Fc region of human IgG1. The C-terminal of the Fc was fused to the VH and VL domains of the CD3 antibody via a glycine-serine linker that will enable the flexibility and folding of the individual domains. This Dual Immune Cell Engager (DICE) includes a binding site for PD1 also at the N-terminal of the second construct. Addition of a PD-1 binding site enhances T-cell activation by blocking the checkpoint inhibition caused by PD-1 and PD-L1 interaction. The Fc region contains three mutations (T366S, T368A, Y407V) that form the hole and another mutation (K392D) that creates an electrostatic steering for proper chain pairing. A second construct containing the VL domain fused to VH domain via a glycine-serine linker followed by Fc region of hIgG1 with a mutation that form the knob was generated (T366W). The human IgG1 Fc includes three mutations (L234A, L235A, P329G) that abrogates the binding to the Fc receptors of hematopoietic cells and C1q, a component of the complement system and thereby it can minimize the secondary immunological reactions such cytokine release syndrome and complement activation. See FIG. 1F.

h7B8-1-hCD3-hPD1 tri-specific antibody. h7B8-1-hCD3-hPD1 is a heterodimer with scFvs brought together via knob-into-hole binding. This construct has bivalent binding site for MUC1 and monovalent binding site for CD3 and monovalent binding site for PD1. Heterodimerization by using knobs-into-hole technology with the indicated mutations (T366S, T368A, Y407V against T366W) in the Fc region. A construct was generated by fusing different domains humanized h7B8-1 and humanized CD3 and PD1 antibodies in the following order: The N-terminal of the construct contains the VH domain of h7B8-1 antibody was fused with VL domain using a glycine-serine linker followed by Fc region of human IgG1. The C-terminal of the Fc was fused to the VL and VH domains of the CD3 antibody via a glycine-serine linker that will enable the flexibility and folding of the individual domains. This Dual Immune Cell Engager (DICE) includes a binding site for PD1 also at the N-terminal of the second construct. The Fc region contains three mutations (T366S, T368A, Y407V) that form the hole and another mutation (K392D) that creates an electrostatic steering for proper chain pairing. A second construct containing the VH domain fused to VL domain via a glycine-serine linker followed by Fc region of hIgG1 with a mutation that form the knob was generated (T366W). The human IgG1 Fc includes three mutations (L234A, L235A, P329G) that abrogates the binding to the Fc receptors of hematopoietic cells and C1q, a component of the complement system and thereby it can minimize the secondary immunological reactions such cytokine release syndrome and complement activation. See FIG. 1G.

h7B8-1-hCD3-hPD1 tri-specific antibody. h7B8-1-hCD3-hPD1 is a heterodimer with scFvs brought together via knob-into-hole binding. This construct has bivalent binding site for MUC1 and monovalent binding site for CD3 and monovalent binding site for PD1. Heterodimerization by using knobs-into-hole technology with the indicated mutations (T366S, T368A, Y407V against T366W) in the Fc region. A construct was generated by fusing different domains humanized h7B8-1 and humanized CD3 and PD1 antibodies in the following order: The N-terminal of the construct contains the VL domain of h7B8-1 antibody was fused with VH domain using a glycine-serine linker followed by Fc region of human IgG1. The C-terminal of the Fc was fused to the VH and VL domains of the CD3 antibody via a glycine-serine linker that will enable the flexibility and folding of the individual domains. This Dual Immune Cell Engager (DICE) includes a binding site for PD1 also at the N-terminal of the second construct. The Fc region contains three mutations (T366S, T368A, Y407V) that form the hole and another mutation (K392D) that creates an electrostatic steering for proper chain pairing. A second construct containing the VL domain fused to VH domain via a glycine-serine linker followed by Fc region of hIgG1 with a mutation that form the knob was generated (T366W). The human IgG1 Fc includes three mutations (L234A, L235A, P329G). See FIG. 1H.

h7B8-1-hCD3 bispecific antibody. h7B8-1-hCD3 is a heterodimer with scFvs brought together via knob-into-hole binding. This construct has bivalent binding site for MUC1 and monovalent binding site for CD3 due to heterodimerization by using knobs-into-hole technology with the indicated mutations (T366S, T368A, Y407V against T366W) in the Fc region. The knobs-into-hole technology applies large amino acids in one chain to create a “knob” and employs smaller amino acids for a corresponding “hole” in the other chain. In addition, electrostatic steering of two oppositely charged heavy chains in combination with the single chain variable fragment (scFv) technology ensures correct chain assembly. A construct was generated by fusing different domains humanized 7B8-1 and humanized CD3 antibodies in the following order: The N-terminal of the construct contains the VH domain of h7B8-1 antibody was fused with VL domain using a glycine-serine linker followed by Fc region of human IgG1. The C-terminal of the Fc was fused to the VL and VH domains of the CD3 antibody via a glycine-serine linker that will enable the flexibility and folding of the individual domains. The Fc region contains three mutations (T366S, T368A, Y407V) that form the hole and another mutation (K392D) that creates an electrostatic steering for proper chain pairing. A second construct containing the VH domain fused to VL domain via a glycine-serine linker followed by Fc region of hIgG1 with a mutation that form the knob was generated (T366W). The human IgG1 Fc includes three mutations (L234A, L235A, P329G) that abrogates the binding to the Fc receptors of hematopoietic cells and C1q, a component of the complement system and thereby it can minimize the secondary immunological reactions such cytokine release syndrome and complement activation. See FIG. 1I.

h3D1-hCD3 bispecific antibody (scFv) This format of bispecific antibody has a single chain variable fragment (scFv) that has one binding site each for MUC1 and CD3 and remains as a monomer due to the indicated mutations. A construct was generated by fusing the VL domain of h7B8-1 with the Fc of human IgG1 followed by adding the VL domain of humanized CD3 antibody. VH domain of h7B8-1 was added to the N-terminal and VH domain of humanized CD3 antibody was added to the C-terminal of the construct using glycine-serine linker on both ends. The Fc of human IgG1 contains mutations (L234A, L235A, P329G) to abrogate the Fc receptor mediated effector mechanism as well as C1q binding. See FIG. 1J.

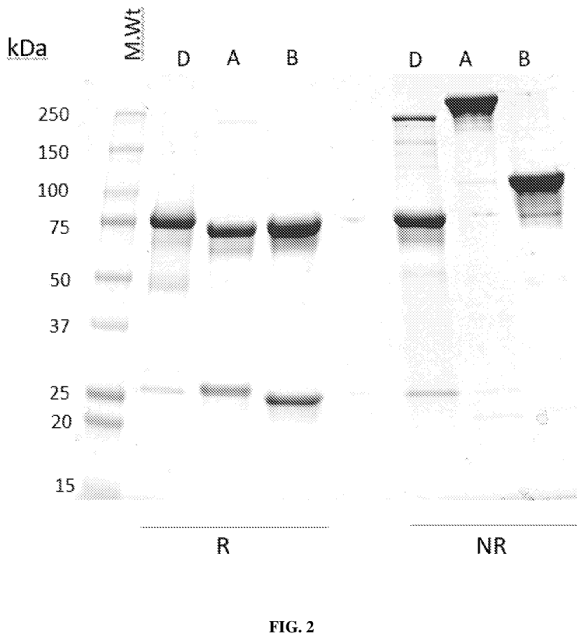

Purification of various bispecific antibodies. All the indicated constructs were expressed in CHO-K1 cells and single cell clones of each bispecific format were generated. Cells from the clones were expanded and suspension cultures were maintained, and the bispecific antibodies were purified using protein A columns. Purified proteins were checked by SDS-PAGE. Lanes 1-3 contain the indicated bispecific proteins in reducing conditions. Lanes 4-6 contain the same proteins in non-reducing conditions. Protein D is a single chain with a molecular weight of 78,500 dalton and presents the same size in the reducing and non-reducing lanes. Protein A has two light chains of 23,515 dalton and a larger fragment of 75,679 dalton each. These bands are seen in the reducing condition of the gel and band of around 200,000 dalton is seen in the non-reducing condition. Protein B has a larger chain of 75,679 dalton and a light chain of 23,885 dalton; they are observed in the reducing condition and a band of 100,000 is observed with the non-reducing condition. These results validate the production of the correct proteins. See FIG. 2.

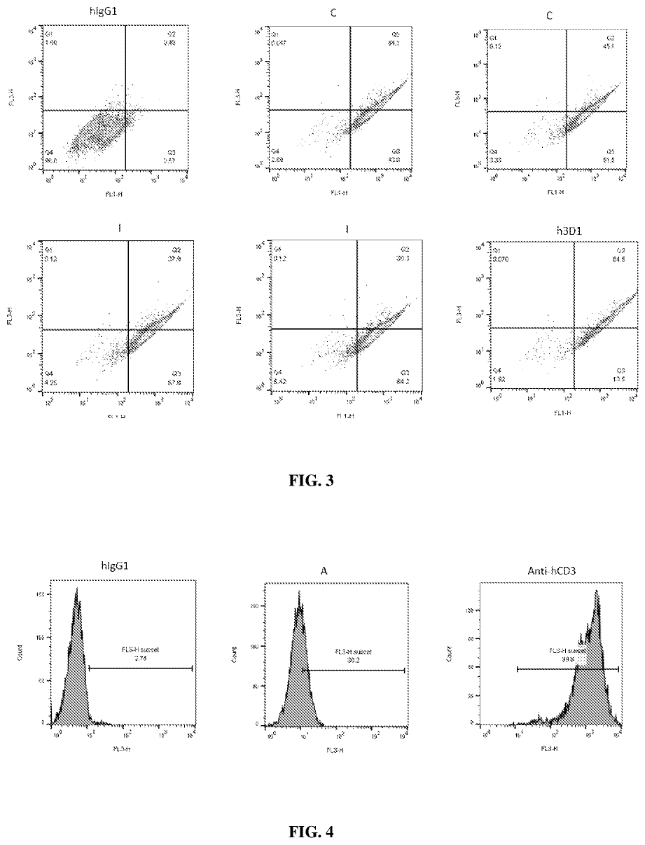

Assessment of h3D1-hCD3 bispecific antibody binding to cell surface MUC1 by Flow Cytometry on breast adenocarcinoma cell line ZR75-1. ZR75-1 cells were harvested and incubated with 1% BSA/PBS for blocking the non-specific binding sites for 20 min and incubated with 4 μg/ml of test antibody (bispecific antibodies) or an IgG1 isotype control antibody. Isotype matched human IgG1 and h3D1 were used as negative and positive control respectively for the binding. After incubation for 60 minutes, cells were washed 2× with PBS. Cells were incubated with appropriate secondary antibody for 45 min and washed 3× with PBS. A fluorescein isothiocyanate (FITC) conjugated Goat F(ab′)2 anti-human immunoglobulin was used as the secondary reagent. Antibody binding to the cell surface was assessed using flow cytometry and the data was analyzed using FlowJo software. See FIG. 3.

Assessment of h3D1-hCD3 bispecific antibody binding to cell surface CD3 by Flow Cytometry on a T cell line, Jurkat. Jurkat cells were harvested and incubated with 1% BSA/PBS for blocking the non-specific binding sites for 20 min and incubated with 4 mg/ml of test antibody (bispecific antibodies) or an IgG1 isotype control antibody. Isotype matched human IgG1 and anti-hCD3 were used as negative and positive control respectively for the binding. After incubation for 60 minutes, cells were washed 2× with PBS. Cells were incubated with appropriate secondary antibody for 45 min and washed 3× with PBS. A fluorescein isothiocyanate (FITC) conjugated Goat F(ab′)2 anti-human immunoglobulin was used as the secondary reagent. Antibody binding to the cell surface was assessed using flow cytometry and the data was analyzed using FlowJo software. See FIG. 4.

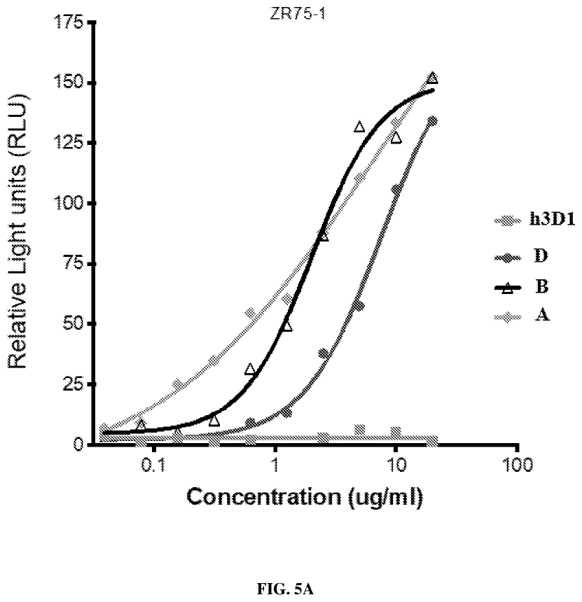

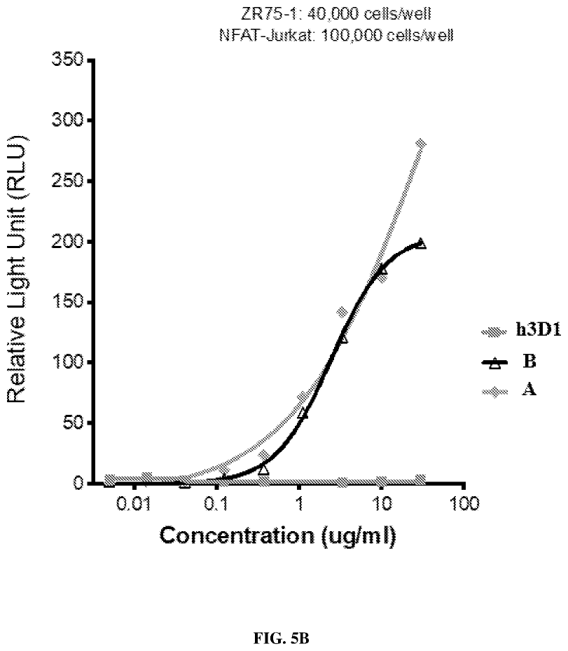

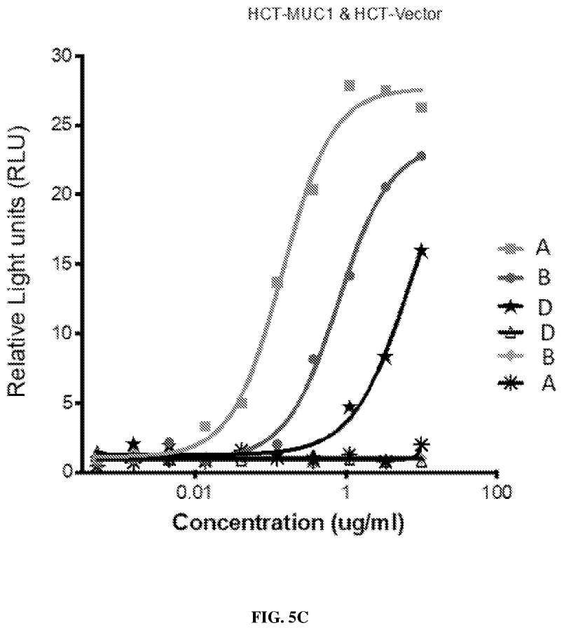

T cell activation by bispecific antibodies in cells endogenously expressing MUC1 (ZR75-1). Target cells (ZR75-1, breast adenocarcinoma cells) were plated in growth medium in a 96 well plate (10,000 cells/well) and incubated overnight. Varying concentrations of bispecific antibodies (D, B or A) starting from 20 ug/ml with 2 fold serial dilution were added to cells followed by TCR/CD3 effector cells (NFAT-Jurkat, 100,000 cells/well) and incubated for 6 hours. Bio-Glo™ Reagent was added and luminescence was quantified using Molecular Devices FilterMax F5 reader. Data were fitted to a 4PL curve using GraphPad Prism software. See FIG. 5A

T cell activation by bispecific antibodies. Target cells (ZR75-1, breast adenocarcinoma cells) were plated in growth medium in a 96 well plate (40,000 cells/well) and incubated overnight. Varying concentrations of bispecific antibodies (B or A) starting from 30 ug/ml with 3 fold serial dilution were added to cells followed by TCR/CD3 effector cells (NFAT-Jurkat, 100,000 cells/well) and incubated for 6 hours. Bio-Glo™ Reagent was added and luminescence was quantified using Molecular Devices FilterMax F5 reader. Data were fitted to a 4PL curve using GraphPad Prism software. See FIG. 5B.

T cell activation by bispecific antibodies in HCT116/vector and HCT116/MUC1 stably expressing cells. HCT116 expressing MUC1 (HCT/MUC1) or the vector (HCT116/Vector) cells (10,000 cells/well) treated with indicated bispecific antibodies (D, B or A) starting from 10 ug/ml with 3 fold serial dilutions and NFAT-Jurkat, 100,000 cells/well and incubated for 6 hours. Bio-Glo™ Reagent was added and luminescence was quantified using Molecular Devices FilterMax F5 reader. Data were fitted to a 4PL curve using GraphPad Prism software. See FIG. 5C.

Binding of the bi-paratopic bi-specific anti-MUC1-C component to MUC1-C antigen. Binding of the bi-paratopic bi-specific anti-MUC1-C component to MUC1-C antigen was measured by ELISA using a positive control (3D1) and medium as negative control. The results are shown in the table below:

TABLE 6
Bi-paratopic bi-specific antibody (design
4) ELISA (to check transfection)

OD

| | | |
|---|---|---|
| Supernatant (3 days) | 2.39 | 2.338 |
| Supernatant (7 days) | 2.06 | 2.335 |
| Medium only | 0.046 | 0.47 |
| positive control 1 ug/ml 3D1 | 2.681 | 2.786 |

### Example 2—Antibody Construct Sequences

1) h3D1(VH-VL)-hFc-hCD3(VL-VH)-scFv
Leader sequence-3D1 heavy chain variable
region-(G4S)3-3D1 Light chain variable region-
G4S-Human IgG1 Fc-G4S-CD3 light chain
variable region-(G4S)3-CD3 heavy chain variable
region:
(SEQ ID NO: 22)
MEFGLSWVFLVALFRGVQCEVQLVQSGAEVKKPGESLKISCKGSG
YAFSNFWMNWVRQMPGKGLEWMGQIYPGDGDTNYNGKFKGQVTIS
ADKSISTAYLQWSSLKASDTAMYYCARSYYRSAWFAYWGQGTLVT
VSLGGGGSGGGGSGGGGSEIVLTQSPDFQSVTPKEKVTITCRASQ
SIGTSIHWYQQKPDQSPKLLIKYASESISGVPSRFSGSGSGTDFT
LTINSLEAEDAATYYCQQSNNWPLTFGQGTKVEIKGGGGSEPKSC
DKTHTCPPCPAPEAAGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALGAPIEKTISKAKGQPREPQVYTLPPSR
DELTKNQVSLSCAVKGFYPSDIAVEWESNGQPENNYDTTPPVLDS
DGSFFLVSKLTVDKSRWQQGNVFSCSVMHEALHNHYTQKSLSLSP
GKGGGGSDIQMTQSPSSLSASVGDRVTITCRASQDIRNYLNWYQQ
KPGKAPKLLIYYTSRLESGVPSRFSGSGSGTDYTLTISSLQPEDF
ATYYCQQGNTLPWTFGQGTKVEIKGGGGSGGGGSGGGGSEVQLVE
SGGGLVQPGGSLRLSCAASGYSFTGYTMNWVRQAPGKGLEWVALI
NPYKGVTTYADSVKGRFTISVDKSKNTAYLQMNSLRAEDTAVYYC
ARSGYYGDSDWYFDVWGQGTLVTVSS
2) h3D1(VH-VL)-hFc-scFv
Leader sequence-3D1 heavy chain variable
region-(G4S)3-3D1 Light chain variable region-
G4S-Human IgG1 Fc:
(SEQ ID NO: 23)
MEFGLSWVFLVALFRGVQCEVQLVQSGAEVKKPGESLKISCKGSG
YAFSNFWMNWVRQMPGKGLEWMGQIYPGDGDTNYNGKFKGQVTIS
ADKSISTAYLQWSSLKASDTAMYYCARSYYRSAWFAYWGQGTLVT
VSLGGGGSGGGGSGGGGSEIVLTQSPDFQSVTPKEKVTITCRASQ
SIGTSIHWYQQKPDQSPKLLIKYASESISGVPSRFSGSGSGTDFT
LTINSLEAEDAATYYCQQSNNWPLTFGQGTKVEIKGGGGSEPKSC
DKTHTCPPCPAPEAAGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALGAPIEKTISKAKGQPREPQVYTLPPSR
DELTKNQVSLWCLVKGFYPSDIA VEWESNGQPENNYKTTPPVLK
SDGSFFLYSKLTVDKSRWQQGNVFSCSVMHEALHNHYTQKSLSLS
PGK
3) h3D1(VL-VH)-hFc-hCD3(VH-VL)-scFv
Leader sequence-3D1 Light chain variable
region-(G4S)3-3D1 heavy chain variable region-
G4S-Human IgG1 Fc-G4S-CD3 heavy chain
variable region-(G4S)3-CD3 light chain variable
region:
(SEQ ID NO: 24)
MKYLLPTAAAGLLLLAAQPAMAEIVLTQSPDFQSVTPKEKVTITC
RASQSIGTSIHWYQQKPDQSPKLLIKYASESISGVPSRFSGSGSG
TDFTLTINSLEAEDAATYYCQQSNNWPLTFGQGTKVEIKGGGGSG
GGGSGGGGSEVQLVQSGAEVKKPGESLKISCKGSGYAFSNFWMNW
VRQMPGKGLEWMGQIYPGDGDTNYNGKFKGQVTISADKSISTAYL
QWSSLKASDTAMYYCARSYYRSAWFAYWGQGTLVTVSLGGGGSEP
KSCDKTHTCPPCPAPEAAGGPSVFLFPPKPKDTLMISRTPEVTCV
VVDVSHEDPEVKFNWYVDGVEVHNAKTKPREEQYNSTYRVVSVLT
VLHQDWLNGKEYKCKVSNKALGAPIEKTISKAKGQPREPQVYTLP
PSRDELTKNQVSLSCAVKGFYPSDIAVEWESNGQPENNYDTTPPV
LDSDGSFFLVSKLTVDKSRWQQGNVFSCSVMHEALHNHYTQKSLS
LSPGKGGGGSEVQLVESGGGLVQPGGSLRLSCAASGYSFTGYTMN
WVRQAPGKGLEWVALINPYKGVTTYADSVKGRFTISVDKSKNTAY
LQMNSLRAEDTAVYYCARSGYYGDSDWYFDVWGQGTLVTVSSGGG
GSGGGGSGGGGSDIQMTQSPSSLSASVGDRVTITCRASQDIRNYL
NWYQQKPGKAPKLLIYYTSRLESGVPSRFSGSGSGTDYTLTISSL
QPEDFATYYCQQGNTLPWTFGQGTKVEIK
4) h3D1(VL-VH)-hFc-scFv
Leader sequence-3D1 Light chain variable
region-(G4S)3-3D1 heavy chain variable region-
G4S-Human IgG1 Fc:
(SEQ ID NO: 25)
MKYLLPTAAAGLLLLAAQPAMAEIVLTQSPDFQSVTPKEKVTITC
RASQSIGTSIHWYQQKPDQSPKLLIKYASESISGVPSRFSGSGSG
TDFTLTINSLEAEDAATYYCQQSNNWPLTFGQGTKVEIKGGGGSG
GGGSGGGGSEVQLVQSGAEVKKPGESLKISCKGSGYAFSNFWMNW
VRQMPGKGLEWMGQIYPGDGDTNYNGKFKGQVTISADKSISTAYL
QWSSLKASDTAMYYCARSYYRSAWFAYWGQGTLVTVSLGGGGSEP
KSCDKTHTCPPCPAPEAAGGPSVFLFPPKPKDTLMISRTPEVTCV
VVDVSHEDPEVKFNWYVDGVEVHNAKTKPREEQYNSTYRVVSVLT
VLHQDWLNGKEYKCKVSNKALGAPIEKTISKAKGQPREPQVYTLP
PSRDELTKNQVSLWCLVKGFYPSDIAVEWESNGQPENNYKTTPPV
LKSDGSFFLYSKLTVDKSRWQQGNVFSCSVMHEALHNHYTQKSLS
LSPGK
5)
h7B8-1(VH-VL)-hFc-hCD3(VL-VH)-scFv
Leader sequence-7B8-1 heavy chain variable
region-(G4S)3-7B8-1 Light chain variable
region-G4S-Human IgG1 Fc-G4S-CD3 light chain
variable region-(G4S)3-CD3 heavy chain
variable region:
(SEQ ID NO: 26)
MEFGLSWVFLVALFRGVQCEVQLVQSGAEVKKPGESLKISCKGSG
FTFNYFWIEWVRQMPGKGLEWMGEILPGTGSTNYNEKFKGQVTIS
ADKSISTAYLQWSSLKASDTAMYYCARYDYTSSMDYWGQGTLVTV
SSGGGGSGGGGSGGGGSEIVLTQSPATLSLSPGERATLSCRASES
VQYSGTSLMHWYQQKPGQAPRLLIYGASNVETGIPARFSGSGSGT
DFTLTISSLEPEDFAVYYCQQNWKVPWTFGQGTKVEIKGGGGSEP
KSCDKTHTCPPCPAPEAAGGPSVFLFPPKPKDTLMISRTPEVTCV
VVDVSHEDPEVKFNWYVDGVEVHNAKTKPREEQYNSTYRVVSVLT
VLHQDWLNGKEYKCKVSNKALGAPIEKTISKAKGQPREPQVYTLP
PSRDELTKNQVSLSCAVKGFYPSDIA VEWESNGQPENNYDTTPP
VLDSDGSFFLVSKLTVDKSRWQQGNVFSCSVMHEALHNHYTQKSL
SLSPGKGGGGSDIQMTQSPSSLSASVGDRVTITCRASQDIRNYLN
WYQQKPGKAPKLLIYYTSRLESGVPSRFSGSGSGTDYTLTISSLQ
PEDFATYYCQQGNTLPWTFGQGTKVEIKGGGGSGGGGSGGGGSEV
QLVESGGGLVQPGGSLRLSCAASGYSFTGYTMNWVRQAPGKGLEW
VALINPYKGVTTYADSVKGRFTISVDKSKNTAYLQMNSLRAEDTA
VYYCARSGYYGDSDWYFDVWGQGTLVTVSS
6)
h7B8-1(VH-VL)-hFc-scFv
Leader sequence-7B8-1 heavy chain variable
region-(G4S)3-7B8-1 Light chain variable
region-G4S-Human IgG1 Fc:
(SEQ ID NO: 27)
MEFGLSWVFLVALFRGVQCEVQLVQSGAEVKKPGESLKISCKGSG
FTFNYFWIEWVRQMPGKGLEWMGEILPGTGSTNYNEKFKGQVTIS
ADKSISTAYLQWSSLKASDTAMYYCARYDYTSSMDYWGQGTLVTV
SSGGGGSGGGGSGGGGSEIVLTQSPATLSLSPGERATLSCRASES
VQYSGTSLMHWYQQKPGQAPRLLIYGASNVETGIPARFSGSGSGT
DFTLTISSLEPEDFAVYYCQQNWKVPWTFGQGTKVEIKGGGGSEP
KSCDKTHTCPPCPAPEAAGGPSVFLFPPKPKDTLMISRTPEVTCV
VVDVSHEDPEVKFNWYVDGVEVHNAKTKPREEQYNSTYRVVSVLT
VLHQDWLNGKEYKCKVSNKALGAPIEKTISKAKGQPREPQVYTLP
PSRDELTKNQVSLWCLVKGFYPSDIAVEWESNGQPENNYKTTPPV
LKSDGSFFLYSKLTVDKSRWQQGNVFSCSVMHEALHNHYTQKSLS
LSPGK
7) h7B8-1(VL-VH)-hFc-CD3
(VH-VL)-scFv
Leader sequence-7B8-1 Light chain variable
region-(G4S)3 7B8-1 heavy chain variable region
-G4S-Human IgG1 Fc-G4S-CD3 heavy chain variable
region-(G4S)3-CD3 light chain
variable region:
(SEQ ID NO: 28)
MKYLLPTAAAGLLLLAAQPAMAEIVLTQSPATLSLSPGERATLSC
RASESVQYSGTSLMHWYQQKPGQAPRLLIYGASNVETGIPARFSG
SGSGTDFTLTISSLEPEDFAVYYCQQNWKVPWTFGQGTKVEIKGG
GGSGGGGSGGGGSEVQLVQSGAEVKKPGESLKISCKGSGFTFNYF
WIEWVRQMPGKGLEWMGEILPGTGSTNYNEKFKGQVTISADKSIS
TAYLQWSSLKASDTAMYYCARYDYTSSMDYWGQGTLVTVSSGGGG
SEPKSCDKTHTCPPCPAPEAAGGPSVFLFPPKPKDTLMISRTPEV
TCVVVDVSHEDPEVKFNWYVDGVEVHNAKTKPREEQYNSTYRVVS
VLTVLHQDWLNGKEYKCKVSNKALGAPIEKTISKAKGQPREPQVY
TLPPSRDELTKNQVSLSCAVKGFYPSDIA VEWESNGQPENNYDT
TPPVLDSDGSFFLVSKLTVDKSRWQQGNVFSCSVMHEALHNHYTQ
KSLSLSPGKGGGGSEVQLVESGGGLVQPGGSLRLSCAASGYSFTG
YTMNWVRQAPGKGLEWVALINPYKGVTTYADSVKGRFTISVDKSK
NTAYLQMNSLRAEDTAVYYCARSGYYGDSDWYFDVWGQGTLVTVS
SGGGGSGGGGSGGGGSDIQMTQSPSSLSASVGDRVTITCRASQDI
RNYLNWYQQKPGKAPKLLIYYTSRLESGVPSRFSGSGSGTDYTLT
ISSLQPEDFATYYCQQGNTLPWTFGQGTKVEIK
8) h7B8-(VL-VH)-hFc-scFv
Leader sequence-7B8-1 Light chain variable region-
(G4S)3-7B8-1 heavy chain variable region
-G4S-Human IgG1 Fc:
(SEQ ID NO: 29)
MKYLLPTAAAGLLLLAAQPAMAEIVLTQSPATLSLSPGERATLSC
RASESVQYSGTSLMHWYQQKPGQAPRLLIYGASNVETGIPARFSG
SGSGTDFTLTISSLEPEDFAVYYCQQNWKVPWTFGQGTKVEIKGG
GGSGGGGSGGGGSEVQLVQSGAEVKKPGESLKISCKGSGFTFNYF
WIEWVRQMPGKGLEWMGEILPGTGSTNYNEKFKGQVTISADKSIS
TAYLQWSSLKASDTAMYYCARYDYTSSMDYWGQGTLVTVSSGGGG
SEPKSCDKTHTCPPCPAPEAAGGPSVFLFPPKPKDTLMISRTPEV
TCVVVDVSHEDPEVKFNWYVDGVEVHNAKTKPREEQYNSTYRVVS
VLTVLHQDWLNGKEYKCKVSNKALGAPIEKTISKAKGQPREPQVY
TLPPSRDELTKNQVSLWCLVKGFYPSDIA VEWESNGQPENNYKT
TPPVLKSDGSFFLYSKLTVDKSRWQQGNVFSCSVMHEALHNHYTQ
KSLSLSPGK
9) h3D1(VH-CH1)-hFc-hCD3(VL-VH)-scFv
VH of humanized anti-MUC-1 antibody 3D1,
CH1 of human IgG1, Fc of human IgG1 with
LALA-PG mutations, Linker = Anti-human CD3 VL,
Linker = Anti-human CD3 VH:
(SEQ ID NO: 30)
EVQLVQSGAEVKKPGESLKISCKGSGYAFSNFWMNWVRQMPGKGL
EWMGQIYPGDGDTNYNGKFKGQVTISADKSISTAYLQWSSLKASD
TAMYYCARSYYRSAWFAYWGQGTLVTVSLASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPEAAGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALGAPIEKTISKAKGQPREPQVYTLPPSRDEL
TKNQVSLTCLVKGFYPSDIA VEWESNGQPENNYKTTPPVLDSDG
SFFLYSKLTVDKSRWQQGNVFSCSVMHEALHNHYTQKSLSLSPGK
GGGGSDIQMTQSPSSLSASVGDRVTITCRASQDIRNYLNWYQQKP
GKAPKLLIYYTSRLESGVPSRFSGSGSGTDYTLTISSLQPEDFAT
YYCQQGNTLPWTFGQGTKVEIKGGGGSGGGGSGGGGSEVQLVESG
GGLVQPGGSLRLSCAASGYSFTGYTMNWVRQAPGKGLEWVALINP
YKGVTTYADSVKGRFTISVDKSKNTAYLQMNSLRAEDTAVYYCAR
SGYYGDSDWYFDVWGQGTLVTVSS
10) h3D1
(VL-CL)
VL of humanized anti-MUC-1 antibody 3D1,
CL of human IgG1:
(SEQ ID NO: 31)
EIVLTQSPDFQSVTPKEKVTITCRASQSIGTSIHWYQQKPDQSPK
LLIKYASESISGVPSRFSGSGSGTDFTLTINSLEAEDAATYYCQQ
SNNWPLTFGQGTKVEIKRTVAAPSVFIFPPSDEQLKSGTASVVCL
LNNFYPRESKVQWKVDNALQSGNSQESVTEQDSKDSTYSLSSTLT
LSKADYEKHKVYACEVTHQGLSSPVTKSFNRGEC
11) h7B8-1(VH-CH1)-mhFc-hCD3 (VL-VH)-scFv
VH of humanized anti-MUC-1 antibody 7B8-1,
CH1 of human IgG1, mhFc of human IgG1,
Linker = Anti-human CD3 VL,
Linker = Anti-human CD3 VH:
(SEQ ID NO: 32)
EVQLVQSGAEVKKPGESLKISCKGSGFTFNYFWIEWVRQMPGKGL
EWMGEILPGTGSTNYNEKFKGQVTISADKSISTAYLQWSSLKASD
TAMYYCARYDYTSSMDYWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKKVEPKSCDKTY
TGPPGPAPELLGGPSVFCFPPKPKDTLMISRTPEVTCVVVDVSHE
DPEVKFNWYVDGVEVHNAKTKPREEQYNSTYRVVSVLTVLHQDWL
NGKEYKCKVSNKALPAPIECTISKAKGQCREPQVYTLPPSRDELT
KNQVSLRCHVKGFYPSDIAVEWESNGQPENNYKTTKPVLDSDGSF
FLYSTLTVDKSRWQQGNVFSCSVLHECLHNHYTQKSLSLSPGKGG
GGSDIQMTQSPSSLSASVGDRVTITCRASQDIRNYLNWYQQKPGK
APKLLIYYTSRLESGVPSRFSGSGSGTDYTLTISSLQPEDFATYY
CQQGNTLPWTFGQGTKVEIKGGGGSGGGGSGGGGSEVQLVESGGG
LVQPGGSLRLSCAASGYSFTGYTMNWVRQAPGKGLEWVALINPYK
GVTTYADSVKGRFTISVDKSKNTAYLQMNSLRAEDTAVYYCARSG
YYGDSDWYFDVWGQGTLVTVSS
12)
h7B8-1 (VL-CL)
VL of humanized anti-MUC-1 antibody 7B8-1,
CL of human IgG1:
(SEQ ID NO: 33)
EIVLTQSPATLSLSPGERATLSCRASESVQYSGTSLMHWYQQKPG
QAPRLLIYGASNVETGIPARFSGSGSGTDFTLTISSLEPEDFA V
YYCQQNWKVPWTFGQGTKVEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
13)
h3D1 (VH-VL)-hFc-hPD-1(VL-VH)-scFv
Leader sequence-3D1 heavy chain variable region-
(G4S)3-3D1 Light chain variable region-
G4S-Human IgG1 Fc-PD1 light chain variable
region-(G4S)3-PD1 heavy chain variable
region:
(SEQ ID NO: 34)
MEFGLSWVFLVALFRGVQCEVQLVQSGAEVKKPGESLKISCKGSG
YAFSNFWMNWVRQMPGKGLEWMGQIYPGDGDTNYNGKFKGQVTIS
ADKSISTAYLQWSSLKASDTAMYYCARSYYRSAWFAYWGQGTLVT
VSLGGGGSGGGGSGGGGSEIVLTQSPDFQSVTPKEKVTITCRASQ
SIGTSIHWYQQKPDQSPKLLIKYASESISGVPSRFSGSGSGTDFT
LTINSLEAEDAATYYCQQSNNWPLTFGQGTKVEIKGGGGSEPKSC
DKTHTCPPCPAPEAAGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALGAPIEKTISKAKGQPREPQVYTLPPSR
DELTKNQVSLWCLVKGFYPSDIAVEWESNGQPENNYKTTPPVLKS
DGSFFLYSKLTVDKSRWQQGNVFSCSVMHEALHNHYTQKSLSLSP
GKGGGGSEIVLTQSPATLSLSPGERATLSCRASKGVSTSGYSYLH
WYQQKPGQAPRLLIYLASYLESGVPARFSGSGSGTDFTLTISSLE
PEDFAVYYCQHSRDLPLTFGGGTKVEIKGGGGSGGGGSGGGGSQV
QLVQSGVEVKKPGASVKVSCKASGYTFTNYYMYWVRQAPGQGLEW
MGGINPSNGGTNFNEKFKNRVTLTTDSSTTTAYMELKSLQFDDTA
VYYCARRDYRFDMGFDYWGQGTTVTVSS
14) h3D1(VL-VH)-hFc-hPD-1(VH-VL)-scFv
Leader sequence-3D1 Light chain variable
region-(G4S)3-3D1 heavy chain variable region-
G4S-Human IgG1 Fc-G4S-PD1 heavy chain
variable region-(G4S)3-PD1 light chain
variable region:
(SEQ ID NO: 35)
MKYLLPTAAAGLLLLAAQPAMAEIVLTQSPDFQSVTPKEKVTITC
RASQSIGTSIHWYQQKPDQSPKLLIKYASESISGVPSRFSGSGSG
TDFTLTINSLEAEDAATYYCQQSNNWPLTFGQGTKVEIKGGGGSG
GGGSGGGGSEVQLVQSGAEVKKPGESLKISCKGSGYAFSNFWMNW
VRQMPGKGLEWMGQIYPGDGDTNYNGKFKGQVTISADKSISTAYL
QWSSLKASDTAMYYCARSYYRSAWFAYWGQGTLVTVSLGGGGSEP
KSCDKTHTCPPCPAPEAAGGPSVFLFPPKPKDTLMISRTPEVTCV
VVDVSHEDPEVKFNWYVDGVEVHNAKTKPREEQYNSTYRVVSVLT
VLHQDWLNGKEYKCKVSNKALGAPIEKTISKAKGQPREPQVYTLP
PSRDELTKNQVSLWCLVKGFYPSDIAVEWESNGQPENNYKTTPPV
LKSDGSFFLYSKLTVDKSRWQQGNVFSCSVMHEALHNHYTQKSLS
LSPGKGGGGSQVQLVQSGVEVKKPGASVKVSCKASGYTFTNYYMY
WVRQAPGQGLEWMGGINPSNGGTNFNEKFKNRVTLTTDSSTTTAY
MELKSLQFDDTAVYYCARRDYRFDMGFDYWGQGTTVTVSSGGGGS
GGGGSGGGGSEIVLTQSPATLSLSPGERATLSCRASKGVSTSGYS
YLHWYQQKPGQAPRLLIYLASYLESGVPARFSGSGSGTDFTLTIS
SLEPEDFAVYYCQHSRDLPLTFGGGTKVEIK
15)
h7B8-1 (VH-VL)-hFc-hPD-1 (VL-VH)-scFv
Leader sequence-7B8 heavy chain variable
region-(G4S)3-7B8 Light chain variable region-
G4S-Human IgG1 Fc-PD1 light chain variable
region-(G4S)3-PD1 heavy chain variable
region:
(SEQ ID NO: 36)
MEFGLSWVFLVALFRGVQCEVQLVQSGAEVKKPGESLKISCKGSG
FTFNYFWIEWVRQMPGKGLEWMGEILPGTGSTNYNEKFKGQVTIS
ADKSISTAYLQWSSLKASDTAMYYCARYDYTSSMDYWGQGTLVTV
SSGGGGSGGGGSGGGGSEIVLTQSPATLSLSPGERATLSCRASES
VQYSGTSLMHWYQQKPGQAPRLLIYGASNVETGIPARFSGSGSGT
DFTLTISSLEPEDFAVYYCQQNWKVPWTFGQGTKVEIKGGGGSEP
KSCDKTHTCPPCPAPEAAGGPSVFLFPPKPKDTLMISRTPEVTCV
VVDVSHEDPEVKFNWYVDGVEVHNAKTKPREEQYNSTYRVVSVLT
VLHQDWLNGKEYKCKVSNKALGAPIEKTISKAKGQPREPQVYTLP
PSRDELTKNQVSLWCLVKGFYPSDIAVEWESNGQPENNYKTTPPV
LKSDGSFFLYSKLTVDKSRWQQGNVFSCSVMHEALHNHYTQKSLS
LSPGKGGGGSEIVLTQSPATLSLSPGERATLSCRASKGVSTSGYS
YLHWYQQKPGQAPRLLIYLASYLESGVPARFSGSGSGTDFTLTIS
SLEPEDFAVYYCQHSRDLPLTFGGGTKVEIKGGGGSGGGGSGGGG
SQVQLVQSGVEVKKPGASVKVSCKASGYTFTNYYMYWVRQAPGQG
LEWMGGINPSNGGTNFNEKFKNRVTLTTDSSTTTAYMELKSLQFD
DTA VYYCARRDYRFDMGFDYWGQGTTVTVSS
16) h7B8-1
(VL-VH)-hFc-hPD-1
(VH-VL)-scFv
Leader sequence-7B8 Light chain variable
region-(G4S)3-7B8 heavy chain variable region-
G4S-Human IgG1 Fc-G4S-PD1 heavy chain
variable region-(G4S)3-PD1 light chain
variable region:
(SEQ ID NO: 37)
MKYLLPTAAAGLLLLAAQPAMAEIVLTQSPATLSLSPGERATLSC
RASESVQYSGTSLMHWYQQKPGQAPRLLIYGASNVETGIPARFSG
SGSGTDFTLTISSLEPEDFAVYYCQQNWKVPWTFGQGTKVEIKGG
GGSGGGGSGGGGSEVQLVQSGAEVKKPGESLKISCKGSGFTFNYF
WIEWVRQMPGKGLEWMGEILPGTGSTNYNEKFKGQVTISADKSIS
TAYLQWSSLKASDTAMYYCARYDYTSSMDYWGQGTLVTVSSGGGG
SEPKSCDKTHTCPPCPAPEAAGGPSVFLFPPKPKDTLMISRTPEV
TCVVVDVSHEDPEVKFNWYVDGVEVHNAKTKPREEQYNSTYRVVS
VLTVLHQDWLNGKEYKCKVSNKALGAPIEKTISKAKGQPREPQVY
TLPPSRDELTKNQVSLWCLVKGFYPSDIAVEWESNGQPENNYKTT
PPVLKSDGSFFLYSKLTVDKSRWQQGNVFSCSVMHEALHNHYTQK
SLSLSPGKGGGGSQVQLVQSGVEVKKPGASVKVSCKASGYTFTNY
YMYWVRQAPGQGLEWMGGINPSNGGTNFNEKFKNRVTLTTDSSTT
TAYMELKSLQFDDTAVYYCARRDYRFDMGFDYWGQGTTVTVSSGG
GGSGGGGSGGGGSEIVLTQSPATLSLSPGERATLSCRASKGVSTS
GYSYLHWYQQKPGQAPRLLIYLASYLESGVPARFSGSGSGTDFTL
TISSLEPEDFAVYYCQHSRDLPLTFGGGTKVEIK
h7B8/h3D1-hCD3 bi-paratopic bi-specific 7B8
(VH-CH1)-Fc-CD3 (VL-VH):
Leader sequence-7B8 Heavy chain variable
region (VH5)-Human IgG1 constant
region-CD3 (VL-VH)
(SEQ ID NO: 38)
MEFGLSWVFLVALFRGVQCEVQLVQSGAEVKKPGESLKISCKGSG
FTFNYFWIEWVRQMPGKGLEWMGEILPGTGSTNYNEKFKGQVTIS
ADKSISTAYLQWSSLKASDTAMYYCARYDYTSSMDYWGQGTLVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPEAAGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALGAPIEKTISKAK
GQPREPQVYTLPPSRDELTKNQVSLSCAVKGFYPSDIAVEWESNG
QPENNYDTTPPVLDSDGSFFLVSDLTVDKSRWQQGNVFSCSVMHE
ALHNHYTQKSLSLSPGKGGGGSDIQMTQSPSSLSASVGDRVTITC
RASQDIRNYLNWYQQKPGKAPKLLIYYTSRLESGVPSRESGSGSG
TDYTLTISSLQPEDFATYYCQQGNTLPWTFGQGTKVEIKGGGGSG
GGGSGGGGSEVQLVESGGGLVQPGGSLRLSCAASGYSFTGYTMNW
VRQAPGKGLEWVALINPYKGVTTYADSVKGRFTISVDKSKNTAYL
QMNSLRAEDTAVYYCARSGYYGDSDWYFDVWGQGTLVTVSS*
7B8
(VH-CH1)-Fc-CD3 (VH-VL):
Leader sequence-7B8 Heavy chain variable
region (VH5)-Human IgG1 constant region-
CD3-CD3 (VH-VL)
(SEQ ID NO: 39)
MEFGLSWVFLVALFRGVQCEVQLVQSGAEVKKPGESLKISCKGSG
FTFNYFWIEWVRQMPGKGLEWMGEILPGTGSTNYNEKFKGQVTIS
ADKSISTAYLQWSSLKASDTAMYYCARYDYTSSMDYWGQGTLVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDKTHTCPPCPAPEAAGGPSVFLFPPKPKDT
LMISRTPEVTCVVVDVSHEDPEVKFNWYVDGVEVHNAKTKPREEQ
YNSTYRVVSVLTVLHQDWLNGKEYKCKVSNKALGAPIEKTISKAK
GQPREPQVYTLPPSRDELTKNQVSLSCAVKGFYPSDIAVEWESNG
QPENNYDTTPPVLDSDGSFFLVSDLTVDKSRWQQGNVFSCSVMHE
ALHNHYTQKSLSLSPGKGGGGSEVQL VESGGGLVQPGGSLRLSC
AASGYSFTGYTMNWVRQAPGKGLEWVALINPYKGVTTYADSVKGR
FTISVDKSKNTAYLQMNSLRAEDTAVYYCARSGYYGDSDWYFDVW
GQGTLVTVSSGGGGSGGGGSGGGGSDIQMTQSPSSLSASVGDRVT
ITCRASQDIRNYLNWYQQKPGKAPKLLIYYTSRLESGVPSRESGS
GSGTDYTLTISSLQPEDFATYYCQQGNTLPWTFGQGTKVEIK*
h7B8 Light Chain (VL-CL):
Leader sequence-7B8 Light chain variable
region (VL3)-Human Ig kappa constant
region
(SEQ ID NO: 40)
MKYLLPTAAAGLLLLAAQPAMAEIVLTQSPATLSLSPGERATLSC
RASESVQYSGTSLMHWYQQKPGQAPRLLIYGASNVETGIPARFSG
SGSGTDFTLTISSLEPEDFAVYYCQQNWKVPWTFGQGTKVEIKRT
VAAPSVFIFPPSDEQLKSGTASVVCLLNNFYPREAKVQWKVDNAL
QSGNSQESVTEQDSKDSTYSLSSTLTLSKADYEKHKVYACEVTHQ
GLSSPVTKSFNRGEC*
3D1 (VH5-VL1)-Fc allotype 2:
Leader sequence-3D1 heavy chain variable
region-(G4S)3 linker-3D1 Light chain variable
region-Human IgG1 Fc
(SEQ ID NO: 41)
MEFGLSWVFLVALFRGVQCEVQLVQSGAEVKKPGESLKISCKGSG
YAFSNFWMNWVRQMPGKGLEWMGQIYPGDGDTNYNGKFKGQVTIS
ADKSISTAYLQWSSLKASDTAMYYCARSYYRSAWFAYWGQGTLVT
VSLGGGGSGGGGSGGGGSEIVLTQSPDFQSVTPKEKVTITCRASQ
SIGTSIHWYQQKPDQSPKLLIKYASESISGVPSRFSGSGSGTDFT
LTINSLEAEDAATYYCQQSNNWPLTFGQGTKVEIKGGGGSEPKSC
DKTYTCPPCPAPEAAGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALGAPIEKTISKAKGQPREPQVYTLPPSR
KEMTKNQVSLWCLVKGFYPSDIA VEWESNGQPENNYKTTPPVLK
SDGSFFLYSKLTVDKSRWQQGNVFSCSVMHEALHNHYTQKSLSLS
PGK
3D1 (VL1-VH5) with Fc allotype 2:
Leader sequence-3D1 Light chain variable
region-(G4S)3-3D1 heavy chain variable region
G4S-Human IgG1 Fc
(SEQ ID NO: 42)
MKYLLPTAAAGLLLLAAQPAMAEIVLTQSPDFQSVTPKEKVTITC
RASQSIGTSIHWYQQKPDQSPKLLIKYASESISGVPSRFSGSGSG
TDFTLTINSLEAEDAATYYCQQSNNWPLTFGQGTKVEIKGGGGSG
GGGSGGGGSEVQLVQSGAEVKKPGESLKISCKGSGYAFSNFWMNW
VRQMPGKGLEWMGQIYPGDGDTNYNGKFKGQVTISADKSISTAYL
QWSSLKASDTAMYYCARSYYRSAWFAYWGQGTLVTVSLGGGGSEP
KSCDKTYTCPPCPAPEAAGGPSVFLFPPKPKDTLMISRTPEVTCV
VVDVSHEDPEVKFNWYVDGVEVHNAKTKPREEQYNSTYRVVSVLT
VLHQDWLNGKEYKCKVSNKALGAPIEKTISKAKGQPREPQVYTLP
PSRKEMTKNQVSLWCLVKGFYPSDIAVEWESNGQPENNYKTTPPV
LKSDGSFFLYSKLTVDKSRWQQGNVFSCSVMHEALHNHYTQKSLS
LSPGK

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,683,202
U.S. Pat. No. 4,684,611
U.S. Pat. No. 4,816,567
U.S. Pat. No. 4,867,973
U.S. Pat. No. 4,879,236
U.S. Pat. No. 4,938,948
U.S. Pat. No. 4,952,500
U.S. Pat. No. 5,021,236
U.S. Pat. No. 5,141,648
U.S. Pat. No. 5,196,066
U.S. Pat. No. 5,217,879
U.S. Pat. No. 5,302,523
U.S. Pat. No. 5,322,783
U.S. Pat. No. 5,384,253
U.S. Pat. No. 5,464,765
U.S. Pat. No. 5,506,138
U.S. Pat. No. 5,538,877
U.S. Pat. No. 5,538,880
U.S. Pat. No. 5,550,318
U.S. Pat. No. 5,563,055
U.S. Pat. No. 5,563,250
U.S. Pat. No. 5,565,332
U.S. Pat. No. 5,580,859
U.S. Pat. No. 5,589,466
U.S. Pat. No. 5,610,042
U.S. Pat. No. 5,656,610
U.S. Pat. No. 5,670,488
U.S. Pat. No. 5,702,932
U.S. Pat. No. 5,736,524
U.S. Pat. No. 5,739,018
U.S. Pat. No. 5,780,448
U.S. Pat. No. 5,789,215
U.S. Pat. No. 5,824,544
U.S. Pat. No. 5,830,725
U.S. Pat. No. 5,849,304
U.S. Pat. No. 5,851,826
U.S. Pat. No. 5,856,456
U.S. Pat. No. 5,858,744
U.S. Pat. No. 5,871,982
U.S. Pat. No. 5,871,983
U.S. Pat. No. 5,871,986
U.S. Pat. No. 5,879,934
U.S. Pat. No. 5,880,270
U.S. Pat. No. 5,888,502
U.S. Pat. No. 5,925,565
U.S. Pat. No. 5,928,906
U.S. Pat. No. 5,932,210
U.S. Pat. No. 5,935,819
U.S. Pat. No. 5,945,100
U.S. Pat. No. 5,955,331
U.S. Pat. No. 5,981,274
U.S. Pat. No. 5,994,136
U.S. Pat. No. 5,994,624
U.S. Pat. No. 6,013,516
U.S. patent Ser. No. 12/789,127
U.S. patent Ser. No. 13/026,858
U.S. patent Ser. No. 13/045,033
“Antibodies: A Laboratory Manual,” Cold Spring Harbor Press, Cold Spring Harbor, N Y, 1988.
Abbondanzo et al., Am. J. Pediatr. Hematol. Oncol., 12(4), 480-489, 1990.
Allred et al., Arch. Surg., 125(1), 107-113, 1990.
Almendro et al., J. Immunol., 157(12):5411-5421, 1996.
Amado and Chen, Science, 285(5428):674-676, 1999.
Angel et al., Cell, 49:729, 1987a.
Angel et al., Cell, 49:729, 1987b.
Armentano et al., Proc. Natl. Acad. Sci. USA, 87(16):6141-6145, 1990.
Atchison and Perry, Cell, 46:253, 1986.
Atchison and Perry, Cell, 48:121, 1987.
Atherton et al., Biol. of Reproduction, 32, 155-171, 1985.
Ausubel et al., Current Protocols in Molecular Biology, Greene Publishing Associates and Wiley Interscience, N.Y., 1994.
Baldus et al., Clin. Cancer Res., 10(8):2790-2796, 2004.
Banerji et al., Cell, 27:299, 1981.
Banerji et al., Cell, 33(3):729-740, 1983.
Bates, Mol. Biotechnol., 2(2):135-145, 1994.
Batra et al., Am. J. Respir. Cell Mol. Biol., 21(2):238-245, 1999.
Battraw and Hall, Theor. App. Genet., 82(2):161-168, 1991.
Beidler et al., J. Immunol., 141(11):4053-4060, 1988.
Berkhout et al., Cell, 59:273-282, 1989.
Bett et al., J. Virololgy, 67(10):5911-5921, 1993.
Bhattacharjee et al., J. Plant Bioch. Biotech., 6(2):69-73. 1997.
Bilbao et al., FASEB J., 11(8):624-634, 1997.
Blackwell et al., Arch. Otolaryngol Head Neck Surg., 125(8):856-863, 1999.
Blanar et al., EMBO J., 8:1139, 1989.
Blomer et al., J. Virol., 71(9):6641-6649, 1997.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Brown et al., J. Immunol. Meth., 12; 130(1), 111-121, 1990.
Bulla and Siddiqui, J. Virol., 62:1437, 1986.
Campbell and Villarreal, Mol. Cell. Biol., 8:1993, 1988.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Campere and Tilghman, Genes and Dev., 3:537, 1989.
Campo et al., Nature, 303:77, 1983.
Capaldi et al., Biochem. Biophys. Res. Comm., 74(2):425-433, 1977.
Caplen et al., Gene Ther., 6(3):454-459, 1999.
Carbonelli et al., FEMS Microbiol. Lett., 177(1):75-82, 1999.
Case et al., Proc. Natl. Acad. Sci. USA, 96(6):2988-2993, 1999.
Celander and Haseltine, J. Virology, 61:269, 1987.
Celander et al., J. Virology, 62:1314, 1988.
Chandler et al., Proc. Natl. Acad. Sci. USA, 94(8):3596-601, 1997.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7(8):2745-2752, 1987.
Chillon et al., J. Virol., 73(3):2537-2540, 1999.
Choi et al., J. Mol. Biol., 262(2):151-167, 1996.
Christou et al., Proc. Natl. Acad. Sci. USA, 84(12):3962-3966, 1987.
Clay et al., J. Immunol., 162:1749, 1999.
Cocea, Biotechniques, 23(5):814-816, 1997.
Coffey et al., Science, 282(5392):1332-1334, 1998.
Cohen et al., J. Cell. Physiol., 5:75, 1987.
Costa et al., Mol. Cell. Biol., 8:81-90, 1988.
Cripe et al., EMBO J., 6:3745, 1987.
Culotta and Hamer, Mol. Cell. Biol., 9:1376-1380, 1989.
D'Halluin et al., Plant Cell, 4(12):1495-1505, 1992.
Dandolo et al., J. Virology, 47:55-64, 1983.
De Jager et al., Semin. Nucl. Med. 23(2), 165-179, 1993.
DeLuca et al., J. Virol., 56(2):558-570, 1985.
Derby et al., Hear Res, 134(1-2):1-8, 1999.
Deschamps et al., Science, 230:1174-1177, 1985.
Detappe et al., Int. J. Radiation Oncol. 108(5):1380-1389, 2020.
Dholakia et al., J. Biol. Chem., 264, 20638-20642, 1989.
Doolittle and Ben-Zeev, Methods Mol. Biol., 109, 215-237, 1999.
Dorai et al., Int. J. Cancer, 82(6):846-52, 1999.
Duraisamy et al., Gene, 373:28-34, 2006.
Edbrooke et al., Mol. Cell. Biol., 9:1908-1916, 1989.
Edlund et al., Science, 230:912-916, 1985.
Engel and Kohn, Front Biosci, 4:e26-33, 1999.
EP Application 125,023
EP Application 171,496
EP Application 173,494
EP Application 184,187
EPO 0273085
Fechheimer et al., Proc Natl. Acad. Sci. USA, 84:8463-8467, 1987.
Feldman et al., Cardiovasc. Res., 32(2):194-207, 1996.
Feldman et al., Semin. Interv. Cardiol., 1(3):203-208, 1996.
Feng and Holland, Nature, 334:6178, 1988.
Feng et al., Nat. Biotechnol., 15(9):866-870, 1997.
Firak and Subramanian, Mol. Cell. Biol., 6:3667, 1986.
Fisher et al., Hum. Gene Ther., 7(17):2079-2087, 1996.
Foecking and Hofstetter, Gene, 45(1):101-105, 1986.
Fraley et al., Proc. Natl. Acad. Sci. USA, 76:3348-3352, 1979.
Fujita et al., Cell, 49:357, 1987.
Fujiwara and Tanaka, Nippon Geka Gakkai Zasshi, 99(7):463-468, 1998.
Garoff and Li, Curr. Opin. Biotechnol., 9(5):464-469, 1998.
Garrido et al., J. Neurovirol., 5(3):280-288, 1999.
Gefter et al., Somatic Cell Genet., 3:231-236, 1977.
Gendler et al., J. Biol. Chem., 263:12820-12823, 1988.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gillies et al., Cell, 33:717, 1983.
Gloss et al., EMBO J., 6:3735, 1987.
Gnant et al., Cancer Res., 59(14):3396-403, 1999.
Gnant et al., J. Natl. Cancer Inst., 91(20):1744-1750, 1999.
Godbout et al., Mol. Cell. Biol., 8:1169, 1988.
Goding, In: Monoclonal Antibodies: Principles and Practice, 2d ed., Orlando, Fla., Academic Press, 60-61, 65-66, 71-74, 1986.
Goodbourn and Maniatis, Cell, 41(2):509-520, 1985.
Goodbourn et al., Cell, 45:601, 1986.
Gopal, Mol. Cell Biol., 5:1188-1190, 1985.
Graham and Prevec, Mol Biotechnol, 3(3):207-220, 1995.
Graham and Van Der Eb, Virology, 52:456-467, 1973.
Greene et al., Immunology Today, 10:272, 1989.
Grosschedl and Baltimore, Cell, 41:885, 1985.
Gulbis and Galand, Hum. Pathol. 24(12), 1271-1285, 1993.
Haecker et al., Hum. Gene Ther., 7(15):1907-1914, 1996.
Han et al., J. Infect. Dis., 179:230-233, 1999.
Harland and Weintraub, J. Cell Biol., 101(3):1094-1099, 1985.
Haslinger and Karin, Proc. Natl. Acad. Sci. USA, 82:8572, 1985.
Hauber and Cullen, J. Virology, 62:673, 1988.
Hayashi et al., Neurosci. Lett., 267(1):37-40, 1999.
He et al., Plant Cell Reports, 14 (2-3):192-196, 1994.
Hen et al., Nature, 321:249, 1986.
Hensel et al., Lymphokine Res., 8:347, 1989.
Hermens and Verhaagen, Prog. Neurobiol., 55(4):399-432, 1998.
Herr and Clarke, Cell, 45:461, 1986.
Hirochika et al., J. Virol., 61:2599, 1987.
Hirsch et al., Mol. Cell. Biol., 10:1959, 1990.
Holbrook et al., Virology, 157:211, 1987.
Holzer et al. Virology, 253(1):107-114, 1999.
Horlick and Benfield, Mol. Cell. Biol., 9:2396, 1989.
Hou and Lin, Plant Physiology, 111:166, 1996.
Howard et al., Ann. NY Acad. Sci., 880:352-365, 1999.
Huang et al., Cell, 27:245, 1981.
Huard et al., Neuromuscul Disord, 7(5):299-313, 1997.
Hug et al., Mol. Cell. Biol., 8:3065-3079, 1988.
Hwang et al., Mol. Cell. Biol., 10:585, 1990.
Imagawa et al., Cell, 51:251, 1987.
Imai et al., Nephrologie, 19(7):397-402, 1998.
Imbra and Karin, Nature, 323:555, 1986.
Imler et al., Mol. Cell. Biol., 7:2558, 1987.
Imperiale and Nevins, Mol. Cell. Biol., 4:875, 1984.
Irie et al., Antisense Nucleic Acid Drug Dev., 9(4):341-349, 1999.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9(8):3393-3399, 1989.
Johnston et al., J. Virol., 73(6):4991-5000, 1999.
Jones et al., Nature, 321:522-525, 1986.
Kadesch and Berg, Mol. Cell. Biol., 6:2593, 1986.
Kaeppler et al., Plant Cell Rep., 8:415-418, 1990.
Kaneda et al., Science, 243:375-378, 1989.
Karin et al., Mol. Cell. Biol., 7:606, 1987.
Karin et al., Mol. Cell. Biol., 7:606, 1987.
Katinka et al., Cell, 20:393, 1980.
Katinka et al., Nature, 290:720, 1981.
Kato et al, J. Biol. Chem., 266:3361-3364, 1991.
Kaufman et al., Arch. Ophthalmol., 117(7):925-928, 1999.
Kawamoto et al., Mol. Cell. Biol., 8:267, 1988.
Kay, Haemophilia, 4(4):389-392, 1998.
Khatoon et al., Ann. of Neurology, 26, 210-219, 1989.
Kiledjian et al., Mol. Cell. Biol., 8:145, 1988.
King et al., J. Biol. Chem., 269, 10210-10218, 1989.
Kinlough et al., J. Biol. Chem., 279(51):53071-53077, 2004.
Kinoshita et al., Biochem. Biophys. Res. Commun., 394:205-210, 2010.
Klamut et al., Mol. Cell. Biol., 10:193, 1990.
Klimatcheva et al., Front Biosci, 4:D481-496, 1999.
Koch et al., Mol. Cell. Biol., 9:303, 1989.
Kohler and Milstein, Eur. J. Immunol., 6, 511-519, 1976.
Kohler and Milstein, Nature, 256, 495-497, 1975.
Kohut et al., Am. J. Physiol., 275(6Ptl):L1089-1094, 1998.
Kooby et al., FASEB J, 13(11):1325-34, 1999.
Kraus et al. FEBS Lett., 428(3):165-170, 1998.
Kriegler and Botchan, Mol. Cell. Biol., 3:325, 1983.
Kriegler et al., Cell, 38:483, 1984a.
Kriegler et al., In: Cancer Cells 2/Oncogenes and Viral Genes, Van de Woude et al. eds, Cold Spring Harbor, Cold Spring Harbor Laboratory, 1984b.
Krisky et al., Gene Ther, 5(11):1517-1530, 1998a.
Krisky et al., Gene Ther, 5(12):1593-1603, 1998b.
Kuhl et al., Cell, 50:1057, 1987.
Kunz et al., Nucl. Acids Res., 17:1121, 1989.
Kyte and Doolittle, J. Mol. Biol., 157(1):105-132, 1982.
Lachmann and Efstathiou, Curr. Opin. Mol. Ther., 1(5):622-632, 1999.
Lareyre et al., J. Biol. Chem., 274(12):8282-8290, 1999.
Larsen et al., Proc. Natl. Acad. Sci. USA, 83:8283, 1986.
Laspia et al., Cell, 59:283, 1989.
Latimer et al., Mol. Cell. Biol., 10:760, 1990.
Lazzeri, Methods Mol. Biol., 49:95-106, 1995.
Lee et al., Environ. Mol. Mutagen., 13(1):54-59, 1989.
Lee et al., Nature, 294:228, 1981.
Lee et al., Nucleic Acids Res., 12:4191-206, 1984.
Lee et al., DNA Cell Biol., 16(11):1267-1275, 1997.
Leibowitz et al., Diabetes, 48(4):745-753, 1999.
Lesch, Biol Psychiatry, 45(3):247-253, 1999.
Levenson et al., Hum. Gene Ther., 9(8):1233-1236, 1998.
Li et al., Cancer Biol. Ther., 2:187-193, 2003b.
Lin et al., Mol. Cell. Biol., 10:850, 1990.
Lundstrom, J. Recept Signal Transduct. Res., 19(1-4):673-686, 1999.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Majors and Varmus, Proc. Natl. Acad. Sci. USA, 80:5866, 1983.
Marienfeld et al., Gene Ther., 6(6):1101-1113, 1999.
Mastrangelo et al., Biotechnol. Bioeng., 65(3):298-305, 1999.
McNeall et al., Gene, 76:81, 1989.
Miksicek et al., Cell, 46:203, 1986.
Miller et al., J. Pharmacol. Exp. Ther., 264:11-16, 1993.
Miyatake et al., Gene Ther., 6:564-572, 1999.
Moldawer et al., Shock, 12(2):83-101, 1999.
Mordacq and Linzer, Genes and Dev., 3:760, 1989.
Moreau et al., Nucl. Acids Res., 9:6047, 1981.
Moriuchi et al., Cancer Res, 58(24):5731-5737, 1998.
Morrison et al., J. Gen. Virol., 78(Pt 4):873-878, 1997.
Morrison, Science, 229(4719):1202-1207, 1985.
Muesing et al., Cell, 48:691, 1987.
Nakamura et al., In: Enzyme Immunoassays: Heterogeneous and Homogeneous Systems, Chapter 27, 1987.
Naldini et al., Science, 272(5259):263-267, 1996.
Neuberger et al., Nucleic Acids Res., 16(14B):6713-6724, 1988.
Neumann et al., Proc. Natl. Acad. Sci. USA, 96(16):9345-9350, 1999.
Ng et al., Nuc. Acids Res., 17:601, 1989.
Nicolau and Sene, Biochim. Biophys. Acta, 721:185-190, 1982.
Nicolau et al., Methods Enzymol., 149:157-176, 1987.
Nomoto et al., Gene, 236(2):259-271, 1999.
Omirulleh et al., Plant Mol. Biol., 21(3):415-428, 1993.
Omitz et al., Mol. Cell. Biol., 7:3466, 1987.
Ondek et al., EMBO J., 6:1017, 1987.
O'Shannessy et al., J. Immun. Meth., 99, 153-161, 1987.
Owens and Haley, J. Biol. Chem., 259, 14843-14848, 1987.
Palmiter et al., Cell, 29:701, 1982.
Parks et al., J. Virol., 71(4):3293-8, 1997.
PCT Application PCT/US86/02269
PCT Application WO 86/01533
PCT Appln. WO 92/17598
PCT Appln. WO 94/09699
PCT Appln. WO 95/06128
Pech et al., Mol. Cell. Biol., 9:396, 1989.
Pelletier and Sonenberg, Nature, 334(6180):320-325, 1988.
Perales et al., Proc. Natl. Acad. Sci. USA, 91:4086-4090, 1994.
Perez-Stable and Constantini, Mol. Cell. Biol., 10:1116, 1990.
Petrof, Eur Respir J, 11(2):492-497, 1998.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Posner et al., Hybridoma 6, 611-625, 1987.
Potrykus et al., Mol. Gen. Genet., 199(2):169-177, 1985.
Potter and Haley, Meth. Enzymol., 91, 613-633, 1983.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Rabinovitch et al., Diabetes, 48(6):1223-1229, 1999.
Reddy et al., Virology, 251(2):414-26, 1998.
Redondo et al., Science, 247:1225, 1990.
Reisman and Rotter, Mol. Cell. Biol., 9:3571, 1989.
Remington's Pharmaceutical Sciences, 15^{th} Ed., 33:624-652, 1990.
Resendez Jr. et al., Mol. Cell. Biol., 8:4579, 1988.
Rhodes et al., Methods Mol. Biol., 55:121-131, 1995.
Rippe et al., Mol. Cell. Biol., 9(5):2224-22277, 1989.
Rippe, et al., Mol. Cell Biol., 10:689-695, 1990.
Rittling et al., Nucl. Acids Res., 17:1619, 1989.
Robbins and Ghivizzani, Pharmacol Ther, 80(1):35-47, 1998.
Robbins et al., Trends Biotechnol., 16(1):35-40, 1998.
Sambrook et al., In: Molecular cloning: a laboratory manual, 2^{nd} Ed., Cold Spring Harbor Laboratory Press, Cold Spring Harbor, N Y, 1989.
Sawai et al. Mol. Genet. Metab., 67(1):36-42, 1999.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Shaw et al., J. Natl. Cancer Inst., 80(19):1553-1559, 1988.
Sherman et al., Mol. Cell. Biol., 9:50, 1989.
Sleigh and Lockett, J. EMBO, 4:3831, 1985.
Smith et al., Neuron., 20:1093-1102, 1998.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stewart et al., Arch. Biochem. Biophys. 365:71-74; 1999.
Stuart et al., Nature, 317:828, 1985.
Sullivan and Peterlin, Mol. Cell. Biol., 7:3315, 1987.
Sun et al., J. Steroid Biochem., 26(1):83-92, 1987.
Suzuki et al., Biochem Biophys Res Commun, 252(3):686-90, 1998.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Timiryasova et al., Int. J. Oncol., 14(5):845-854, 1999.
Treisman, Cell, 42:889, 1985.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tsukada et al., Plant Cell Physiol., 30(4)599-604, 1989.
Tsumaki et al., J. Biol. Chem., 273(36):22861-22864, 1998.
Tur-Kaspa et al., Mol. Cell Biol., 6:716-718, 1986.
Tyndall et al., Nuc. Acids. Res., 9:6231, 1981.
Vanderkwaak et al., Gynecol Oncol, 74(2):227-234, 1999.
Vasseur et al., Proc. Natl. Acad. Sci. USA, 77:1068, 1980.
Verhoeyen et al., Science, 239(4847):1534-1536, 1988.
Wagner et al., Proc. Natl. Acad. Sci. USA 87(9):3410-3414, 1990.
Wang and Calame, Cell, 47:241, 1986.
Wang et al., Infect. Immun., 66:4193-202, 1998.
Wawrzynczak & Thorpe, Cancer Treat Res., 37:239-51, 1988.
Weber et al., Cell, 36:983, 1984.
Wei et al., Cancer Cell, 7:167-178, 2005.
Weihl et al., Neurosurgery, 44(2):239-252, 1999.
Wen et al., J. Biol. Chem., 278:38029-38039, 2003.
White et al. J. Virol., 73(4):2832-2840, 1999.
Wilson, J. Clin. Invest., 98(11):2435, 1996.
Winoto and Baltimore, Cell, 59:649, 1989.
Wong et al., Gene, 10:87-94, 1980.
Wood et al., J. Clin. Lab. Immunol., 17(4):167-171, 1985.
Wu and Wu, Adv. Drug Delivery Rev., 12:159-167, 1993.
Wu and Wu, J. Biol. Chem., 262:4429-4432, 1987.
Wu et al., Biochem. Biophys. Res. Commun., 233(1):221-226, 1997.
Wu et al., Cancer Res., 58(8): 1605-8, 1998.
Yamada et al., Brain Res., 833(2):302-307, 1999.
Yeung et al., Gene Ther., 6(9):1536-1544, 1999.
Yoon et al., J. Gastrointest. Surg., 3(1):34-48, 1999.
Yutzey et al. Mol. Cell. Biol., 9:1397, 1989.
Zhao-Emonet et al., Biochim. Biophys. Acta, 1442(2-3):109-119, 1998.
Zheng et al., J. Gen. Virol., 80(Pt 7):1735-1742, 1999.
Zhou et al., Nature, 361(6412):543-547, 1993.
Zufferey et al., Nat. Biotechnol., 15(9):871-875, 1997.

1. A recombinant antibody construct that binds selectively to MUC1-C extracellular domain (MUC1-C/ECD) defined by SEQ ID NO: 2, wherein said antibody construct also binds to:
(a) CD3;
(b) CD16;
(c) CD28;
(d) myeloid specific antigen;
(e) ErbB2;
(f) EGFR;
(g) CD3 and PD1;
(h) CD16 and PD1;
(i) CD47;
(j) SIRPα;
(k) NKG2D, or
(l) Siglec 9.

2. The antibody construct of claim 1, wherein said antibody construct is divalent.

3. The antibody construct of claim 1, wherein said antibody construct is trivalent.

4. The antibody construct of claim 1, wherein said antibody construct is tetravalent.

5. The antibody construct of claim 1, wherein said antibody construct has two distinct binding specificities for MUC1-C/ECD binding.

6. The antibody construct of claim 1, wherein said antibody construct has MUC1 binding specificity arising from heavy CDR1, CDR2 and CDR3 sequences of SEQ ID NOs: 3, 5, and 7, respectively, and light chain CDR1, CDR2 and CDR3 sequences of SEQ ID NOS; 4, 6, and 8, respectively; and/or MUC1 binding specificity arising from heavy CDR1, CDR2 and CDR3 sequences of SEQ ID NOs: 9, 11, and 13, respectively, and light chain CDR1, CDR2 and CDR3 sequences of SEQ ID NOS; 10, 12, and 14, respectively.

7. The antibody construct of claim 1, wherein said antibody construct contains one or more mutations permitting two distinct antibody chains to lock.

8. The antibody construct of claim 7, wherein said antibody construct contains IgG sequences.

9. The antibody construct of claim 1, wherein antibody construct is a humanized version of a murine antibody.

10. The antibody construct of claim 9, wherein said humanized antibody construct contains IgG sequences.

11. The antibody construct of claim 1, wherein said antibody construct further comprises a label.

12. The antibody construct of claim 11, wherein said label is a peptide tag, an enzyme, a magnetic particle, a chromophore, a fluorescent molecule, a chemiluminescent molecule, or a dye.

13. The antibody construct of claim 1, wherein said antibody construct further comprises an antitumor drug linked thereto.

14. The antibody of claim 13, wherein said antitumor drug is linked to said antibody construct through a photolabile linker.

15. The antibody construct of claim 13, wherein said antitumor drug is linked to said antibody construct through an enzymatically-cleaved linker.

16. The antibody construct of claim 13, wherein said antitumor drug is a toxin, a radioisotope, a cytokine or an enzyme.

17. The antibody construct of claim 1, wherein said antibody construct comprises a sequence of SEQ ID NOS: 22-42.

18. The antibody of claim 1, wherein said antibody construct comprises a sequence having 80%, 85%, 90%, 95% or 99% homology to SEQ ID NOS: 22-42.

19. The antibody construct of claim 1, wherein said antibody construct is conjugated to a nanoparticle or a liposome.

20. The antibody construct of claim 1, wherein induction of cell death comprises antibody-dependent cell cytotoxicity or complement-mediated cytotoxicity.

21. A method of treating cancer comprising contacting a MUC1-positive cancer cell in a subject with the antibody construct of claim 1.

22. The method of claim 21, wherein said MUC1-positive cancer cell is a solid tumor cell.

23. The method of claim 22, wherein said solid tumor cell is a lung cancer cell, brain cancer cell, head & neck cancer cell, breast cancer cell, skin cancer cell, liver cancer cell, pancreatic cancer cell, stomach cancer cell, colon cancer cell, rectal cancer cell, uterine cancer cell, cervical cancer cell, ovarian cancer cell, testicular cancer cell, skin cancer cell, or esophageal cancer cell.

24. The method of claim 21, wherein said MUC1-positive cancer cell is a leukemia or myeloma.

25. The method of claim 24, wherein said leukemia or myeloma is acute myeloid leukemia, chronic myelogenous leukemia or multiple myeloma.

26. The method of claim 21, further comprising contacting said MUC1-positive cancer cell with a second anti-cancer agent or treatment.

27. The method of claim 26, wherein said second anti-cancer agent or treatment is selected from chemotherapy, radiotherapy, immunotherapy, hormonal therapy, or toxin therapy.

28. The method of claim 26, wherein said second anti-cancer agent or treatment inhibits an intracellular MUC1 function.

29. The method of claim 26, wherein said second anti-cancer agent or treatment is given at the same time as said antibody construct.

30. The method of claim 26, wherein said second anti-cancer agent or treatment is given before and/or after said antibody construct.

31. The method of claim 21, wherein said MUC1-positive cancer cell is a metastatic cancer cell, a multiply drug resistant cancer cell or a recurrent cancer cell.

32. The method of claim 21, wherein said antibody results in the induction of cell death, such as by antibody-dependent cell cytotoxicity or complement-mediated cytotoxicity.

33. A cell expressing the antibody construct of claim 1.


SEQUENCE LISTING (42 sequences)

SEQ ID NO: 1 - protein, 158 aa, Homo sapiens
SVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 2 - protein, 58 aa, Artificial sequence
  Synthetic amino acid
SVVVQLTLAFREGTINVHDVETQFNQYKTEAASRYNLTISDVSVSDVPFPFSAQSGAG

SEQ ID NO: 3 - protein, 8 aa, Artificial sequence
  Synthetic amino acid
GFTFNYFW

SEQ ID NO: 4 - protein, 16 aa, Artificial sequence
  Synthetic amino acid
CRASESVQYSGTSLMH

SEQ ID NO: 5 - protein, 8 aa, Artificial sequence
  Synthetic amino acid
ILPGTGST

SEQ ID NO: 6 - protein, 7 aa, Artificial sequence
  Synthetic amino acid
GASNVET

SEQ ID NO: 7 - protein, 10 aa, Artificial sequence
  Synthetic amino acid
RYDYTSSMDY

SEQ ID NO: 8 - protein, 9 aa, Artificial sequence
  Synthetic amino acid
QQNWKVPWT

SEQ ID NO: 9 - protein, 5 aa, Artificial sequence
  Synthetic amino acid
NFWMN

SEQ ID NO: 10 - protein, 11 aa, Artificial sequence
  Synthetic amino acid
RASQSIGTSIH

SEQ ID NO: 11 - protein, 17 aa, Artificial sequence
  Synthetic amino acid
QIYPGDGDTNYNGKFKG

SEQ ID NO: 12 - protein, 7 aa, Artificial sequence
  Synthetic amino acid
YASESIS

SEQ ID NO: 13 - protein, 10 aa, Artificial sequence
  Synthetic amino acid
SYYRSAWFAY

SEQ ID NO: 14 - protein, 9 aa, Artificial sequence
  Synthetic amino acid
QQSNNWPLT

SEQ ID NO: 15 - protein, 4 aa, Artificial sequence
  Synthetic amino acid
CQCR

SEQ ID NO: 16 - protein, 5 aa, Artificial sequence
  Synthetic amino acid
CQCRR

SEQ ID NO: 17 - protein, 6 aa, Artificial sequence
  Synthetic amino acid
CQCRRR

SEQ ID NO: 18 - protein, 7 aa, Artificial sequence
  Synthetic amino acid
CQCRRRR

SEQ ID NO: 19 - protein, 6 aa, Artificial sequence
  Synthetic amino acid
CQCRRK

SEQ ID NO: 20 - protein, 7 aa, Artificial sequence
  Synthetic amino acid
CQCRRKN

SEQ ID NO: 21 - protein, 16 aa, Artificial sequence
  Synthetic amino acid
RRRRRRRRRCQCRRKN

SEQ ID NO: 22 - protein, 746 aa, Artificial sequence
  Synthetic amino acid
MEFGLSWVFLVALFRGVQCEVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLGGGGSGGGGSGGGGSEIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYDTTPPVLDSDGSFFLVSKLTVDKSRWQQGNVFSCSVMHEALHNHYTQKSLSLSPGKGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSS

SEQ ID NO: 23 - protein, 497 aa, Artificial sequence
  Synthetic amino acid
MEFGLSWVFLVALFRGVQCEVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLGGGGSGGGGSGGGGSEIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLKSDGSFFLYSKLTVDKSRWQQGNVFSCSVMHEALHNHYTQKSLSLSPGK

SEQ ID NO: 24 - protein, 749 aa, Artificial sequence
  Synthetic amino acid
MKYLLPTAAAGLLLLAAQPAMAEIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKGGGGSGGGGSGGGGSEVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYDTTPPVLDSDGSFFLVSKLTVDKSRWQQGNVFSCSVMHEALHNHYTQKSLSLSPGKGGGGSEVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 25 - protein, 500 aa, Artificial sequence
  Synthetic amino acid
MKYLLPTAAAGLLLLAAQPAMAEIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKGGGGSGGGGSGGGGSEVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLKSDGSFFLYSKLTVDKSRWQQGNVFSCSVMHEALHNHYTQKSLSLSPGK

SEQ ID NO: 26 - protein, 749 aa, Artificial sequence
  Synthetic amino acid
MEFGLSWVFLVALFRGVQCEVQLVQSGAEVKKPGESLKISCKGSGFTFNYFWIEWVRQMPGKGLEWMGEILPGTGSTNYNEKFKGQVTISADKSISTAYLQWSSLKASDTAMYYCARYDYTSSMDYWGQGTLVTVSSGGGGSGGGGSGGGGSEIVLTQSPATLSLSPGERATLSCRASESVQYSGTSLMHWYQQKPGQAPRLLIYGASNVETGIPARFSGSGSGTDFTLTISSLEPEDFAVYYCQQNWKVPWTFGQGTKVEIKGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYDTTPPVLDSDGSFFLVSKLTVDKSRWQQGNVFSCSVMHEALHNHYTQKSLSLSPGKGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSS

SEQ ID NO: 27 - protein, 500 aa, Artificial sequence
  Synthetic amino acid
MEFGLSWVFLVALFRGVQCEVQLVQSGAEVKKPGESLKISCKGSGFTFNYFWIEWVRQMPGKGLEWMGEILPGTGSTNYNEKFKGQVTISADKSISTAYLQWSSLKASDTAMYYCARYDYTSSMDYWGQGTLVTVSSGGGGSGGGGSGGGGSEIVLTQSPATLSLSPGERATLSCRASESVQYSGTSLMHWYQQKPGQAPRLLIYGASNVETGIPARFSGSGSGTDFTLTISSLEPEDFAVYYCQQNWKVPWTFGQGTKVEIKGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLKSDGSFFLYSKLTVDKSRWQQGNVFSCSVMHEALHNHYTQKSLSLSPGK

SEQ ID NO: 28 - protein, 752 aa, Artificial sequence
  Synthetic amino acid
MKYLLPTAAAGLLLLAAQPAMAEIVLTQSPATLSLSPGERATLSCRASESVQYSGTSLMHWYQQKPGQAPRLLIYGASNVETGIPARFSGSGSGTDFTLTISSLEPEDFAVYYCQQNWKVPWTFGQGTKVEIKGGGGSGGGGSGGGGSEVQLVQSGAEVKKPGESLKISCKGSGFTFNYFWIEWVRQMPGKGLEWMGEILPGTGSTNYNEKFKGQVTISADKSISTAYLQWSSLKASDTAMYYCARYDYTSSMDYWGQGTLVTVSSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYDTTPPVLDSDGSFFLVSKLTVDKSRWQQGNVFSCSVMHEALHNHYTQKSLSLSPGKGGGGSEVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 29 - protein, 503 aa, Artificial sequence
  Synthetic amino acid
MKYLLPTAAAGLLLLAAQPAMAEIVLTQSPATLSLSPGERATLSCRASESVQYSGTSLMHWYQQKPGQAPRLLIYGASNVETGIPARFSGSGSGTDFTLTISSLEPEDFAVYYCQQNWKVPWTFGQGTKVEIKGGGGSGGGGSGGGGSEVQLVQSGAEVKKPGESLKISCKGSGFTFNYFWIEWVRQMPGKGLEWMGEILPGTGSTNYNEKFKGQVTISADKSISTAYLQWSSLKASDTAMYYCARYDYTSSMDYWGQGTLVTVSSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLKSDGSFFLYSKLTVDKSRWQQGNVFSCSVMHEALHNHYTQKSLSLSPGK

SEQ ID NO: 30 - protein, 698 aa, Artificial sequence
  Synthetic amino acid
EVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSS

SEQ ID NO: 31 - protein, 214 aa, Artificial sequence
  Synthetic amino acid
EIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKRTVAAPSVFIFPPSDEQLKSGTASVVCLLNNFYPRESKVQWKVDNALQSGNSQESVTEQDSKDSTYSLSSTLTLSKADYEKHKVYACEVTHQGLSSPVTKSFNRGEC

SEQ ID NO: 32 - protein, 697 aa, Artificial sequence
  Synthetic amino acid
EVQLVQSGAEVKKPGESLKISCKGSGFTFNYFWIEWVRQMPGKGLEWMGEILPGTGSTNYNEKFKGQVTISADKSISTAYLQWSSLKASDTAMYYCARYDYTSSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTYTGPPGPAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQCREPQVYTLPPSRDELTKNQVSLRCHVKGFYPSDIAVEWESNGQPENNYKTTKPVLDSDGSFFLYSTLTVDKSRWQQGNVFSCSVLHECLHNHYTQKSLSLSPGKGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSS

SEQ ID NO: 33 - protein, 218 aa, Artificial sequence
  Synthetic amino acid
EIVLTQSPATLSLSPGERATLSCRASESVQYSGTSLMHWYQQKPGQAPRLLIYGASNVETGIPARFSGSGSGTDFTLTISSLEPEDFAVYYCQQNWKVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 748 aa, Artificial sequence
  Synthetic amino acid
MEFGLSWVFLVALFRGVQCEVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLGGGGSGGGGSGGGGSEIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLKSDGSFFLYSKLTVDKSRWQQGNVFSCSVMHEALHNHYTQKSLSLSPGKGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 35 - protein, 751 aa, Artificial sequence
  Synthetic amino acid
MKYLLPTAAAGLLLLAAQPAMAEIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKGGGGSGGGGSGGGGSEVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLKSDGSFFLYSKLTVDKSRWQQGNVFSCSVMHEALHNHYTQKSLSLSPGKGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 36 - protein, 751 aa, Artificial sequence
  Synthetic amino acid
MEFGLSWVFLVALFRGVQCEVQLVQSGAEVKKPGESLKISCKGSGFTFNYFWIEWVRQMPGKGLEWMGEILPGTGSTNYNEKFKGQVTISADKSISTAYLQWSSLKASDTAMYYCARYDYTSSMDYWGQGTLVTVSSGGGGSGGGGSGGGGSEIVLTQSPATLSLSPGERATLSCRASESVQYSGTSLMHWYQQKPGQAPRLLIYGASNVETGIPARFSGSGSGTDFTLTISSLEPEDFAVYYCQQNWKVPWTFGQGTKVEIKGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLKSDGSFFLYSKLTVDKSRWQQGNVFSCSVMHEALHNHYTQKSLSLSPGKGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 37 - protein, 754 aa, Artificial sequence
  Synthetic amino acid
MKYLLPTAAAGLLLLAAQPAMAEIVLTQSPATLSLSPGERATLSCRASESVQYSGTSLMHWYQQKPGQAPRLLIYGASNVETGIPARFSGSGSGTDFTLTISSLEPEDFAVYYCQQNWKVPWTFGQGTKVEIKGGGGSGGGGSGGGGSEVQLVQSGAEVKKPGESLKISCKGSGFTFNYFWIEWVRQMPGKGLEWMGEILPGTGSTNYNEKFKGQVTISADKSISTAYLQWSSLKASDTAMYYCARYDYTSSMDYWGQGTLVTVSSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLKSDGSFFLYSKLTVDKSRWQQGNVFSCSVMHEALHNHYTQKSLSLSPGKGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 38 - protein, 716 aa, Artificial sequence
  Synthetic antibody
MEFGLSWVFLVALFRGVQCEVQLVQSGAEVKKPGESLKISCKGSGFTFNYFWIEWVRQMPGKGLEWMGEILPGTGSTNYNEKFKGQVTISADKSISTAYLQWSSLKASDTAMYYCARYDYTSSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYDTTPPVLDSDGSFFLVSDLTVDKSRWQQGNVFSCSVMHEALHNHYTQKSLSLSPGKGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSS

SEQ ID NO: 39 - protein, 716 aa, Artificial sequence
  Synthetic antibody
MEFGLSWVFLVALFRGVQCEVQLVQSGAEVKKPGESLKISCKGSGFTFNYFWIEWVRQMPGKGLEWMGEILPGTGSTNYNEKFKGQVTISADKSISTAYLQWSSLKASDTAMYYCARYDYTSSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYDTTPPVLDSDGSFFLVSDLTVDKSRWQQGNVFSCSVMHEALHNHYTQKSLSLSPGKGGGGSEVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 40 - protein, 240 aa, Artificial sequence
  Synthetic antibody
MKYLLPTAAAGLLLLAAQPAMAEIVLTQSPATLSLSPGERATLSCRASESVQYSGTSLMHWYQQKPGQAPRLLIYGASNVETGIPARFSGSGSGTDFTLTISSLEPEDFAVYYCQQNWKVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 497 aa, Artificial sequence
  Synthetic antibody
MEFGLSWVFLVALFRGVQCEVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLGGGGSGGGGSGGGGSEIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKGGGGSEPKSCDKTYTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKEMTKNQVSLWCLVKGFYPSDIAVEWESNGQPENNYKTTPPVLKSDGSFFLYSKLTVDKSRWQQGNVFSCSVMHEALHNHYTQKSLSLSPGK

SEQ ID NO: 42 - protein, 500 aa, Artificial sequence
  Synthetic antibody
MKYLLPTAAAGLLLLAAQPAMAEIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKGGGGSGGGGSGGGGSEVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLGGGGSEPKSCDKTYTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKEMTKNQVSLWCLVKGFYPSDIAVEWESNGQPENNYKTTPPVLKSDGSFFLYSKLTVDKSRWQQGNVFSCSVMHEALHNHYTQKSLSLSPGK